(19) United States
(12) Patent Application Publication
(10) Pub. No.: US 2006/0014704 A1
(43) Pub. Date: Jan. 19, 2006

(54) Compounds and their preparation for the treatment of Alzheimer's disease by inhibiting beta-amyloid peptide production

(76) Inventors: Donald W. Landry, New York, NY (US); Tae-Wan Kim, Old Tappen, NJ (US); Shixian Deng, White Plains, NY (US)

Correspondence Address: BROWN RAYSMAN MILLSTEIN FELDER & STEINER LLP, 900 THIRD AVENUE, NEW YORK, NY, 10022, US

(21) Appl. No.: 10/961,346

(22) Filed: Oct. 7, 2004

Related U.S. Application Data
(60) Provisional application No. 60/588,433, filed on Jul. 16, 2004.

(51) Int. Cl. C07J 17/00; A61K 31/704

(52) U.S. Cl. 514/26; 536/6.1

(57) ABSTRACT

The present invention provides novel ginsenoside compounds, compositions (e.g. pharmaceutical compositions) comprising the ginsenoside compounds, and methods for the synthesis of these ginsenoside compounds. Additionally, the present invention provides methods for inhibiting beta-amyloid peptide production and methods for treating or preventing a pathological condition, particularly, neurodegeneration diseases (e.g. Alzheimer's disease), using these ginsenoside compounds.

### RELATED APPLICATION

This application claims the benefit of U.S. Provisional Application No. 60/588,433 filed Jul. 16, 2004.

### STATEMENT OF GOVERNMENT INTEREST

This invention was made in part with government support under NIH Grant No. ROI N543467. As such, the United States government may have certain rights in this invention.

### FIELD OF THE INVENTION

The present invention provides novel ginsenoside compounds, compositions (e.g. pharmaceutical compositions) comprising the ginsenoside compounds, and methods for the synthesis of these ginsenoside compounds. Additionally, the present invention provides methods for inhibiting beta-amyloid peptide production and methods for treating or preventing a pathological condition, particularly, neurodegeneration diseases (e.g. Alzheimer's disease), using these ginsenoside compounds.

### BACKGROUND OF THE INVENTION

Alzheimer's disease (AD) is a neurodegenerative disease characterized by a progressive, inexorable loss of cognitive function (Francis, et al., Neuregulins and ErbB receptors in cultured neonatal astrocytes. J. Neurosci. Res., 57:487-94, 1999) that eventually leads to an inability to maintain normal social and/or occupational performance. Alzheimer's disease is the most common form of age-related dementia, and one of the most serious health problems, in the United States. Approximately 4 million Americans suffer from Alzheimer's disease, at an annual cost of at least $100 billion—making Alzheimer's disease one of the costliest disorders of aging. Alzheimer's disease is about twice as common in women as in men, and accounts for more than 65% of the dementias in the elderly. Alzheimer's disease is the fourth leading cause of death in the United States. To date, a cure for Alzheimer's disease is not available, and cognitive decline is inevitable. Although the disease can last for as many as 20 years, AD patients usually live from 8 to 10 years, on average, after being diagnosed with the disease.

The pathogenesis of Alzheimer's disease is associated with an excessive amount of neurofibrillary tangles (composed of paired helical filaments and tau proteins) and neuritic or senile plaques (composed of neurites, astrocytes, and glial cells around an amyloid core) in the cerebral cortex. While senile plaques and neurofibrillary tangles occur with normal aging, they are much more prevalent in persons with Alzheimer's disease. Specific protein abnormalities also occur in Alzheimer's disease. In particular, AD is characterized by the deposition of the amyloid β-peptide (Aβ) into amyloid plaques in the brain (Selkoe, et al. (2001) Alzheimer's disease: genes, proteins, and therapy. Physiol Rev. 81, 741-66; Hardy and Selkoe (2002). The amyloid hypothesis of Alzheimer's disease: progress and problems on the road to therapeutics. Science 297, 2209). Aβ is produced by sequential proteolytic cleavages of amyloid precursor protein (APP) by a set of membrane-bound proteases termed β- and γ-secretases (Vassar and Citron (2000) Abeta-generating enzymes: recent advances in beta- and gamma-secretase research. Neuron 27, 419-422; John, et al. (2003) Human beta-secretase (BACE) and BACE inhibitors. J. Med. Chem. 46, 4625-4630; Selkoe and Kopan (2003) Notch and Presenilin: regulated intramembrane proteolysis links development and degeneration. Annu. Rev Neurosci. 26, 565-597; Medina and Dotti (2003) ripped out by presenilin-dependent gamma-secretase. Cell Signal 15, 829-841). Heterogeneous β-secretase cleavage at the C-terminal end of Aβ produces two major isoforms of Aβ, Aβ40 and Aβ42. While Aβ40 is the predominant cleavage product, the less abundant, highly amyloidogenic Aβ42 is believed to be one of the key pathogenic agents in AD (Selkoe (2001) Alzheimer's disease: genes, proteins, and therapy. Physiol Rev. 81, 741-66) and increased cerebrocorical Aβ42 is closely related to synaptic/neuronal dysfunction associated with AD (Selkoe, Alzheimer's disease is a synaptic failure, Science 298:789-791, 2002).

Presenilins are required for intramembrane proteolysis of selected type-I membrane proteins, including amyloid-beta precursor protein (APP), to yield amyloid-beta protein (De Strooper et al., Deficiency of presenilin-1 inhibits the normal cleavage of amyloid precursor protein. Nature 391:387-90, 1998; Steiner and Haass, Intramembrane proteolysis by presenilins. Nat. Rev. Mol. Cell. Biol. 1:217-24, 2000; Ebinu and Yankner, A rip tide in neuronal signal transduction. Neuron 34:499-502, 2002; De-Strooper and Annaert, Presenilins and the intramembrane proteolysis of proteins: facts and fiction. Nat. Cell Biol. 3:E221-25, 2001; Sisodia and George-Hyslop, γ-Secretase, Notch, α-beta and Alzheimer's disease: where do the presenilins fit in? Nat. Rev. Neurosci. 3:281-90, 2002). Such proteolysis may be mediated by presenilin-dependent β-secretase machinery, which is known to be highly conserved across species, including nematodes, flies, and mammals (L'Hernault and Arduengo, Mutation of a putative sperm membrane protein in Caenorhabditis elegans prevents sperm differentiation but not its associated meiotic divisions. J. Cell. Biol. 119:55-58, 1992; Levitan and Greenwald, Facilitation of lin-12-mediated signaling by sel-12, a Caenorhabditis elegans S182 Alzheimer's disease gene. Nature 377:351-54, 1999; Li and Greenwald, HOP-1, a Caenorhabditis elegans presenilin, appears to be functionally redundant with SEL-12 presenilin and to facilitate LIN-12 and GLP-1 signaling. Proc. Natl. Acad. Sci. USA 94:12204-209, 1997; Steiner and Haass, Intramembrane proteolysis by presenilins. Nat. Rev. Mol. Cell. Biol. 1:217-24, 2000; Sisodia and George-Hyslop, γ-Secretase, Notch, α-beta and Alzheimer's disease: where do the presenilins fit in? Nat. Rev. Neurosci. 3:281-90, 2002).

γ-Secretase, a high-molecular-weight, multi-protein complex harboring presenilin heterodimers and nicastrin, mediates the final step in Aβ production in Alzheimer's disease (Li, et al., Presenilin 1 is linked with β-secretase activity in the detergent solubilized state. Proc. Natl. Acad. Sci. USA 97:6138-43, 2000; Esler, et al., Activity-dependent isolation of the presenilin-γ-secretase complex reveals nicastrin and a gamma substrate. Proc. Natl. Acad. Sci. USA 99:2720-25, 2002). The stabilization of presenilin heterodimers (converted from a short-lived pool to a long-lived pool) and other undefined core components appears to be critical for γ-secretase activity (Thinakaran, et al., Evidence that levels of presenilins (PS1 and PS2) are coordinately regulated by competition for limiting cellular factors. J. Biol. Chem. 272:28415-422, 1997; Tomita, et al., The first proline of PALP motif at the C terminus of presenilins is obligatory for stabilization, complex formation, and gamma-secretase activities of presenilins. J. Biol. Chem. 276:33273-281, 2001). γ-Secretase activity displays very loose sequence specificity near the target transmembrane cleavage site and has been shown to mediate the intramembrane cleavage of other non-APP type-I membrane substrates, including Notch (Schroeter, E. H., et al. (1998) Notch-1 signaling requires ligand-induced proteolytic release of intracellular domain. Nature 393, 382-386; De Strooper, et al. (1999) Presenilin-1-dependent gamma-secretase-like protease mediates release of Notch intracellular domain. Nature 398:518-522), ErbB4 (Lee, et al. (2002) Presenilin-dependent gamma-secretase-like intramembrane cleavage of ErbB4. J. Biol. Chem. 277, 6318-6323; Ni, et al. (2001) Gamma-Secretase cleavage and nuclear localization of ErbB-4 receptor tyrosine kinase. Science 294, 2179-2181), and p75 neurotrophin receptor (p75NTR) (Jung, et al. (2003) Regulated intramembrane proteolysis of the p75 neurotrophin receptor modulates its association with the TrkA receptor. J. Biol. Chem. 278, 42161-42169). It is predicted that general blockage of β-secretase activity not only abolishes Aβ generation but also inhibits normal processing of other cellular β-secretase substrates, required for the relevant cellular function of these substrates. Thus, complete inhibition of γ-secretase activity could potentially lead to severe side-effects (Doerfler, et al., Links Free in PMC Presenilin-dependent gamma-secretase activity modulates thymocyte development. (2001) Proc Natl. Acad. Sci USA 98, 9312-9317; Hadland, et al. Gamma-secretase inhibitors repress thymocyte development. Proc Natl. Acad. Sci USA 98, 7487-7491). A safer approach would ideally be to use reagents which can selectively reduce Aβ42 generation without affecting the intramembrane proteolysis of other γ-secretase substrates. As an example, a subset of nonsteroidal anti-inflammatory drugs (NSAIDs) was shown to decrease the production of Aβ42 (Weggen, et al. (2001). A subset of NSAIDs lower amyloidogenic Abeta42 independently of cyclooxygenase activity. Nature 414, 212-216), without significantly affecting γ-secretase-mediated cleavage of ErbB4 (Weggen, et al. (2003). Abeta42-lowering nonsteroidal anti-inflammatory drugs preserve intramembrane cleavage of the amyloid precursor protein (APP) and ErbB-4 receptor and signaling through the APP intracellular domain. J. Biol. Chem. 278, 30748-30754). Accordingly, small molecules which are able to selectively reduce Aβ42 production (without affecting the cleavage of other γ-secretase substrates) are attractive and promising as therapeutic reagents for treating AD.

Most cases of early-onset familial Alzheimer's disease (FAD) are caused by mutations in two related genes encoding presenilin proteins: PS1 and PS2 (Tanzi, et al., The gene defects responsible for familial Alzheimer's disease. Neurobiol. Dis. 3:159-68, 1996; Hardy, J., Amyloid, the presenilins and Alzheimer's disease. Trends Neurosci. 20:154-59, 1997; Selkoe, D. J., Alzheimer's disease: genes, proteins, and therapy. Physiol. Rev. 81:741-66, 2001). FAD-associated mutations in the presenilins give rise to an increased production of a longer (42 amino acid residues), more amyloidogenic form of amyloid-beta (Aβ42). Deciphering the pathobiology associated with the presenilins provides a unique opportunity to elucidate a molecular basis for Alzheimer's disease. It is suspected that excess beta-amyloid production causes the neuronal degeneration underlying dementia characteristic of AD.

Ginseng is the common name given to the dried roots of plants of the genus Panax which has been used extensively in Asia for thousands of years as a general health tonic and medicine for treating an array of diseases (Cho, et al. (1995) Pharmacological action of Korean ginseng. In the Society for Korean Ginseng (eds.): Understanding Korean Ginseng, Seoul: Hanlim Publishers, pp 35-54; Shibata S. (2001) Chemistry and cancer preventing activities of ginseng saponins and some related triterpenoid compounds. J Korean Med Sci. 16 Suppl:S28-37; Attele, et al. (1999); Ginseng pharmacology: multiple constituents and multiple actions. Biochem Pharmacol. 58:1685-1693; Coleman, et al. (2003). The effects of Panax ginseng on quality of life. J. Clin. Pharm. Ther. 28, 5-15; Coon and Ernst (2002). Panax ginseng: a systematic review of adverse effects and drug interactions. Drug Saf. 25:323-44). The Panax genus contains about six species native to eastern Asia and two species native to eastern North America. Panax ginseng (Asian ginseng) and Panax quinquefolius L. (North American ginseng) are the two species most commonly used in nutraceutical and pharmaceutical compositions. The roots and their extracts contain a variety of substances including saponins.

Ginseng has been well known to have specific pharmacological effects including improvement of liver function and immune enhancement, as well as anti-arteriosclerotic, anti-thrombotic, anti-stress, anti-diabetic, anti-hypertensive and antitumor effects. Among several classes of compounds isolated from the ginseng root, ginseng saponins are known to be the chemical constituents that contribute to its pharmacological effects. These compounds are triterpene glycosides named ginsenosides Rx (x is index “a” to “k” depending on its polarity). The polarity is determined by their mobility on thin-layer chromatography plates and is a function of the number of monosaccharide residues in the molecule's sugar chain.

To date, at least 31 ginsenosides have been isolated from white and red ginseng. All of the ginsenosides can be divided into three groups depending on their aglycons: protopanaxadiol-type ginsenosides (e.g., Rb1, Rb2, Rc, Rd, (20R)Rg3, (20S)Rg3, Rh2), protopanaxatriol-type ginsenosides (e.g., Re, Rf, Rg1, Rg2, Rh1), and oleanolic acid-type ginsenosides (e.g., Ro). Both protopanaxadiol-type and protopanaxatriol-type ginsenosides have a triterpene backbone structure, known as dammarane (Attele, et al. (1999) Ginseng pharmacology: multiple constituents and multiple actions. Biochem. Pharmacol. 58:1685-1693). Rk1, Rg5 (20R)Rg3 and (20S)Rg3 are ginsenosides that are almost uniquely present in heat-processed ginseng, but not found to exist as trace elements in unprocessed ginseng (Kwon, et al. (2001) Liquid chromatographic determination of less polar ginsenosides in processed ginseng. J. Chromatogr. A. 921:335-339; Park, et al. (2002); Cytotoxic dammarane glycosides from processed ginseng. Chem. Pharm. Bul. 50, 538-540 Park, et al. (2002); Three new dammarane glycosides from heat-processed ginseng. Arch. Pharm. Res. 25, 428-432; Kim, et al. (2000); Steaming of ginseng at high temperature enhances biological activity. J. Nat. Prod. 63:1702-1702). Carbohydrates including glucopyranosyl, arabinopyranosyl, arabinofuranosyl and rhamnopyranosyl may also be chemically associated with a particular ginsenoside.

Processing of ginseng with steam at high temperature further enhances the content of these unique ginsenosides Rk1, Rg5, (20R)Rg3 and (20S)Rg3, which appear to possess novel pharmacological activities. At least some of the beneficial qualities of ginseng can be attributed to its triterpene saponin content, a mixture of glucosides referred to collectively as ginsenosides.

U.S. Pat. No. 5,776,460 (“the '460 patent”) discloses a processed ginseng product having enhanced pharmacological effects. This ginseng product, commercially known as “sun ginseng,” contains increased levels of effective pharmacological components due to heat-treating of the ginseng at a high temperature for a particular period of time. As specifically disclosed in the '460 patent, heat treatment of ginseng may be performed at a temperature of 120° to 180° C. for 0.5 to 20 hours, and is preferably performed at a temperature of 120° to 140° C. for 2 to 5 hours. The heating time varies depending on the heating temperature such that lower heating temperatures require longer heating times while higher heating temperatures require comparatively shorter heating times. The '460 patent also discloses that the processed ginseng product has pharmacological properties specifically including anti-oxidant activity and vasodilation activity.

Recently, Tae-Wan Kim et al. demonstrated that the unique components of the heat-processed ginseng product disclosed in the '460 patent significantly lower the production Aβ42 in cells (patent application pending). Specifically, the inventors discovered that at least three ginsenosides Rk1, (20S)Rg3, and Rg5, unique components of the heat-processed ginseng known as “Sun Ginseng,” as well as Rgk351, which is a mixture of (20R)Rg3, (20S)Rg3, Rg5, and Rk1, lower the production of Aβ42 in mammalian cells. Rgk351 and Rk1 are most effective in reducing Aβ42 levels. Furthermore, Rk1 was also shown to inhibit the Aβ42 production in a cell-free assay using a partially purified γ-secretase complex, suggesting that Rk1 modulates either specificity and/or activity of the γ-secretase enzyme. In addition, Tae-Wan Kim et al. found that certain ginsenosides which harbor no Aβ42-reducing activity in vitro, are effective in reducing Aβ42 in vivo. For example, some of the 20(S)-protopanaxatriol (PPT) group ginsenosides, such as Rg1, can be converted into PPT after oral ingestion. Thus, while Rg1 generally has no amyloid-reducing activity in vitro, Rg1 may be converted into an active amyloid-reducing compound PPT in vivo.

### SUMMARY OF THE INVENTION

The present invention provides compositions and methods for preventing and treating neurodegenerative diseases, such as Alzheimer's disease.

In one aspect, the present invention provides a compound having the general formula: 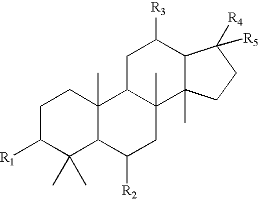
wherein R₁ is selected from the group consisting of α-OH, β-OH, α-O—X, β-O—X, α-R₆COO—, —R₆COO—, α-R₆PO₃—, and β-R₆PO₃—, wherein X is a carbohydrate containing one or more sugars or acylated derivatives thereof, and R₆ is an alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is an alkenyl, aryl, or alkyl II; and R₅ is H or OH. The alkyl I group may further contains oxygen, nitrogen, or phosphorus and the alkyl II group may further contain a functional group selected from the group consisting of hydroxyl, ether, ketone, oxime, hydrazone, imine, and Schiff base. In one embodiment, the sugar group is selected from the group consisting of Glc, Ara(pyr), Ara(fur), Rha, and Xyl. In another embodiment, R₄ is selected from the group consisting of: 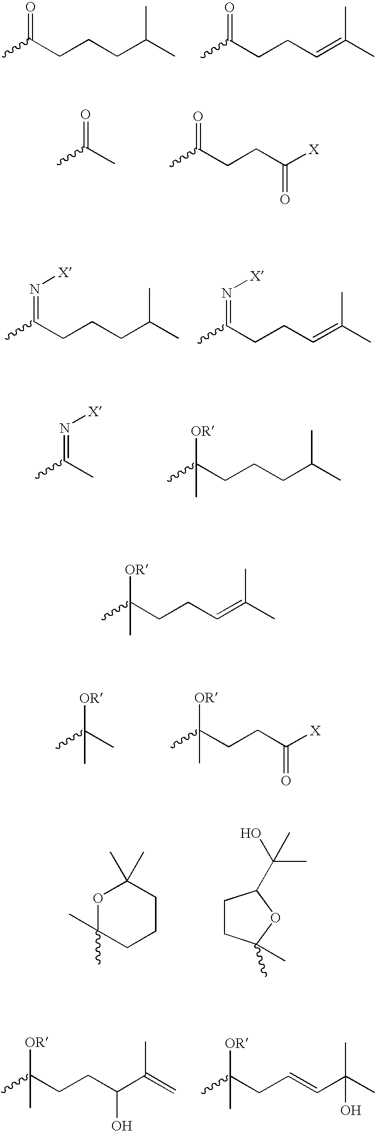
wherein the configuration of any stereo-center is R or S; X is OR or NR, wherein R is alkyl or aryl; X′ is alkyl, OR, or NR, wherein R is alkyl or aryl; and R′ is H, alkyl, or acyl. In another embodiment, the present invention provides a composition, particularly, a pharmaceutical composition, comprising a compound having the general formula: 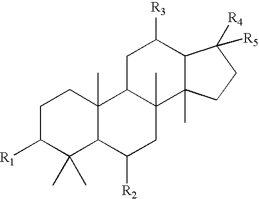
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

The present invention also provides a method for the synthesis of a compound having formula: 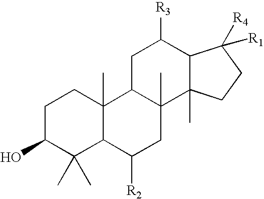
which comprises the steps of:
(a) treating a compound having formula: 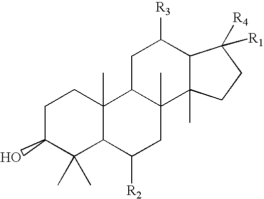
   with an oxidizing agent, to form a compound having formula: 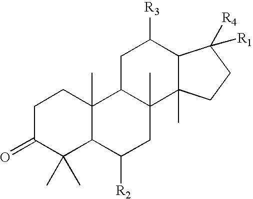
(b) treating the compound formed in step (a) with a reducing agent, to form a compound having formula: 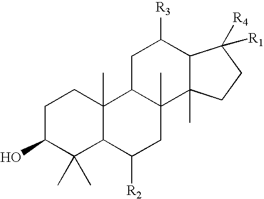
   wherein R₁ is H or OH; R₂ is selected from the group consisting of H, OH, OAc, and O—X, wherein X is a carbohydrate containing one or more sugars or acylated derivatives thereof; R₃ is selected from the group consisting of H, OH, and OAc; and R₄ is alkenyl, aryl, or alkyl. In one embodiment, the oxidizing agent is chromic anhydride and the reducing agent is NaBH₄.

The present invention further provides a method for the synthesis of a compound having formula: 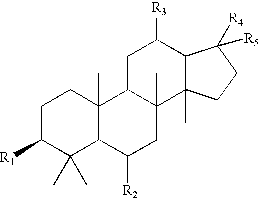
which comprises the steps of:
(a) treating a compound having formula: 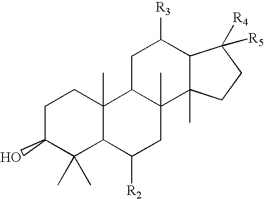
   with an oxidizing agent, to form a compound having formula: 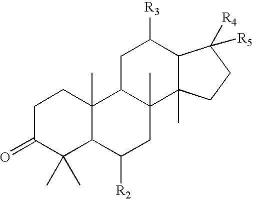
(b) treating the compound formed in step (a) with a reducing agent, to form a compound having formula: 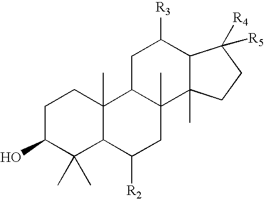
(c) optionally, treating the compound formed in step (b) with protected R₁ derivative, to form a compound having formula: 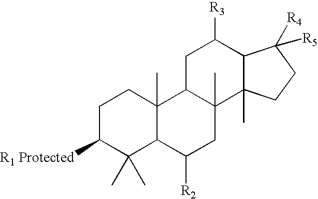
(d) treating the compound formed in step (c) with deprotection agent, to form a compound having formula: 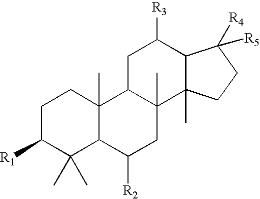
   wherein R1 is selected from the group consisting of α-OH, β-OH, α-O—X, β-O—X, α-R₆COO—, —R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof, R₃ is selected from the group consisting of H, OH, and OAc; R₄ is alkenyl, aryl, or alkyl II; and R₅ is H or OH.

Additionally, the invention provides a method for the synthesis of a compound having formula: 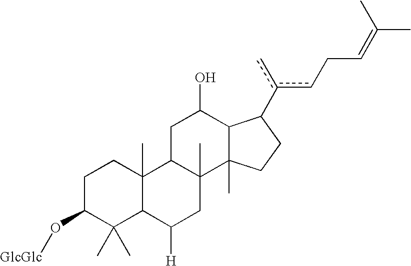
wherein the method comprises the steps of:
(a) treating a compound having formula: 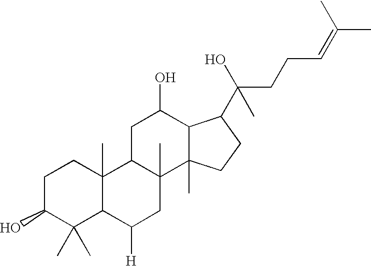
   with an oxidizing agent, to form a compound having formula: 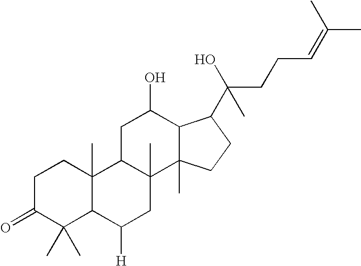
(b) treating the compound formed in step (a) with a protecting agent, to form a compound having formula: 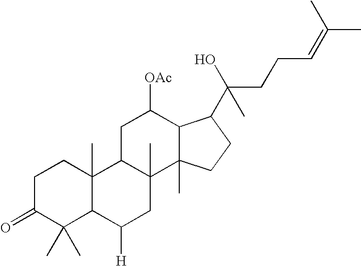
(c) treating the compound formed in step (b) with a reducing agent, to form a compound having formula: 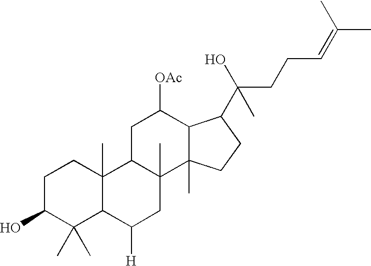
(d) treating the compound formed in step (c) with Ac₈-Glc-Glc-Br, to form a compound having formula: 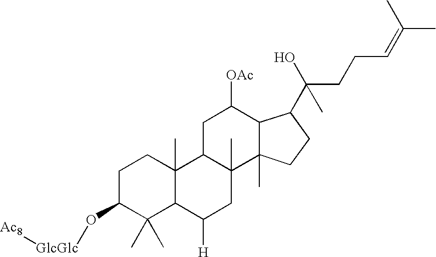
(e) treating the compound formed in step (d) with deprotection agent, to form a compound having formula: 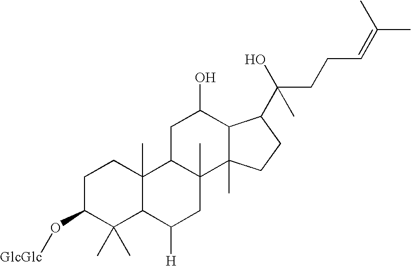
(f) further modifying the compound formed in step (e) to form a compound having formula: 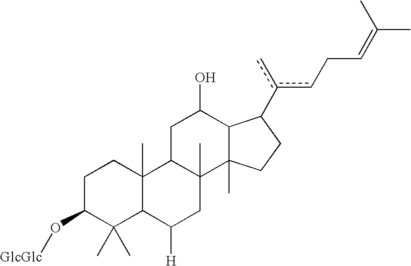 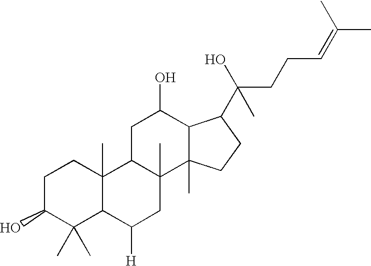
   In one embodiment, the starting material, betulafolienetriol, is obtained from a plant, such as, for example, common birch.

In one aspect, the present invention provides a method for the synthesis of a compound having formula: 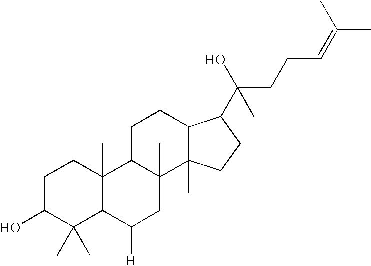
wherein the method comprises the step of treating a compound having formula: 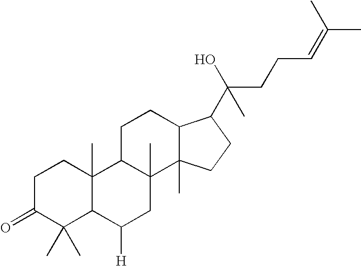
with a reducing agent, such as NaBH₄.

In another aspect, the present invention provides a method for the synthesis of a compound having formula: 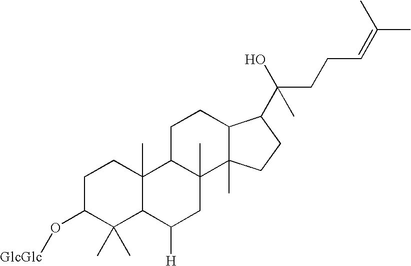
wherein the method comprises the steps of:
(a) treating a compound having formula: 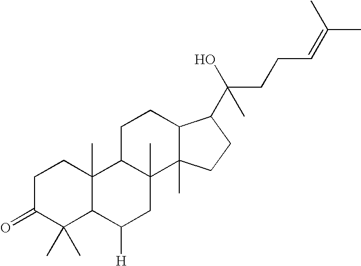
   with a reducing agent, to form a compound having formula: 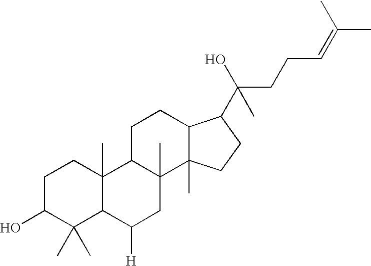
(b) treating the compound formed in step (a) with Ac₈-Glc-Glc-Br, to form a compound having formula: 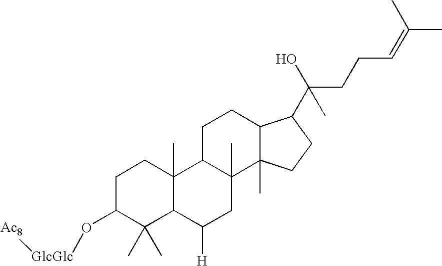
(c) treating the compound formed in step (d) with deprotection agent, to form a compound having formula:

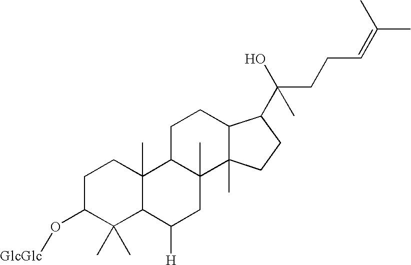

Additionally, the present invention provides a method for treating or preventing a pathological condition in a subject, comprising administering a compound having the general formula: 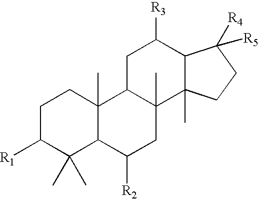
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH. In one embodiment, the pathological condition is neurodegeneration, preferably, Alzheimer's disease and Aβ42-related disorder.

The present invention further provides a method for inhibiting β-amyloid production in subject, including inhibiting β-amyloid production in an in vitro context, comprising administering a compound having the general formula: 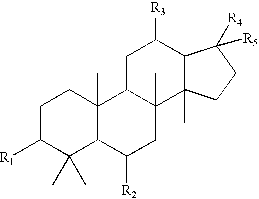
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

Additional aspects of the present invention will be apparent in view of the description that follows.

### BRIEF DESCRIPTION OF THE FIGURES

FIG. 1 depicts sequential proteolytic processing of β-amyloid precursor protein (APP), mediated by β- and γ-secretases.

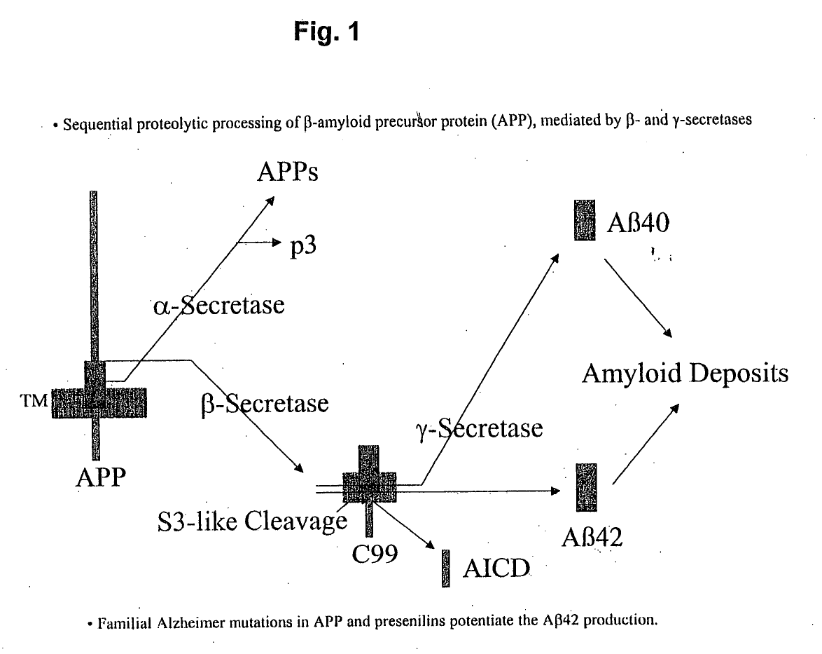

FIG. 2 shows the HPLC profile of (a) White Ginseng; (b) Red Ginseng; and (c) Sun Ginseng (heat processed ginseng).

FIG. 3 illustrates the general chemical formula of: (a) Rg3, (b) Rk1 and (c) Rg5.

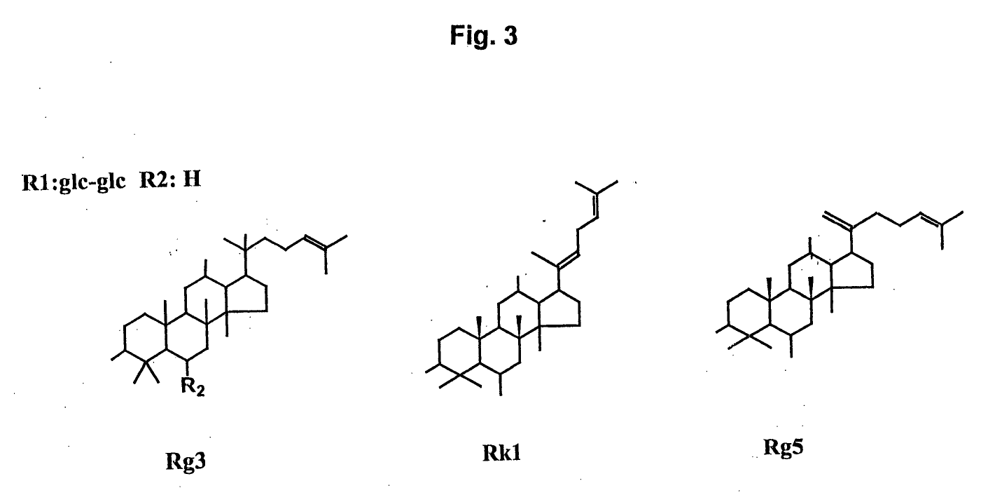

FIG. 4 shows that Rgk351, (20R)Rg3, Rk1 and Rg5 reduce the generation of Aβ42 in CHO cells stably transfected with human APP695. The CHO cells were treated with the indicated compounds (at 50 μg/ml) for 8 hrs. Aβ42 levels in the medium were measured by ELISA and normalized to intracellular full-length APP.

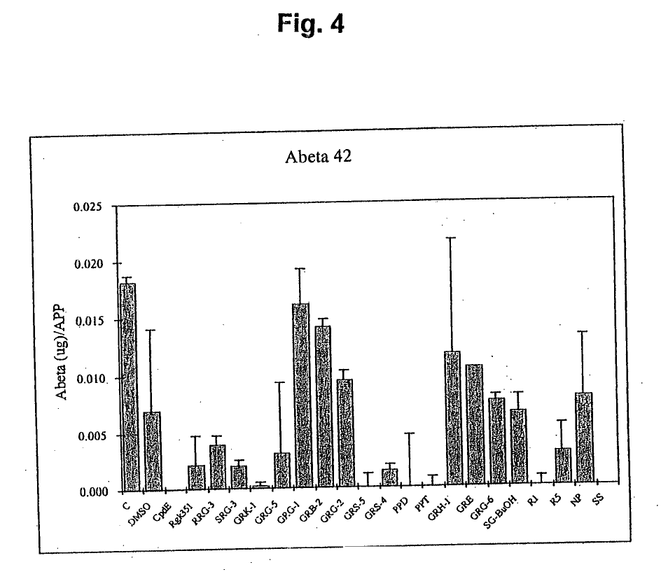

FIG. 5 shows that treatment with Rgk351, Rk1 and Rg5 reduced Aβ42 in the medium of CHO cells expressing human APP in a dose-dependent manner.

FIG. 6 demonstrates that treatment of Rgk351, Rk1 and Rg5 preferentially reduced Aβ42 (vs. Aβ40) in the medium of CHO cells expressing human APP in a dose-dependent manner. The relative levels of Aβ and Aβ42 were normalized to values obtained from non-treated and vehicle-treated cells. Similar data were obtained using Neuro2a-sw (mouse Neuro2a cells expressing Swedish familial Alzheimer's disease mutant form of APP) and 293 cells expressing human APP.

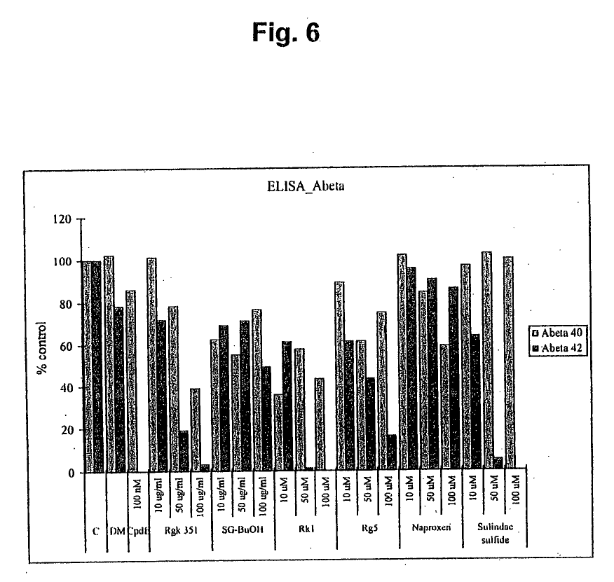

FIG. 7 depicts an analysis of cell lysates and shows that Rgk351, Rk1 and Rg5 caused the increased accumulation of APP C-terminal fragments (γ-secretase substrates), while the full-length holoAPP levels were not affected.

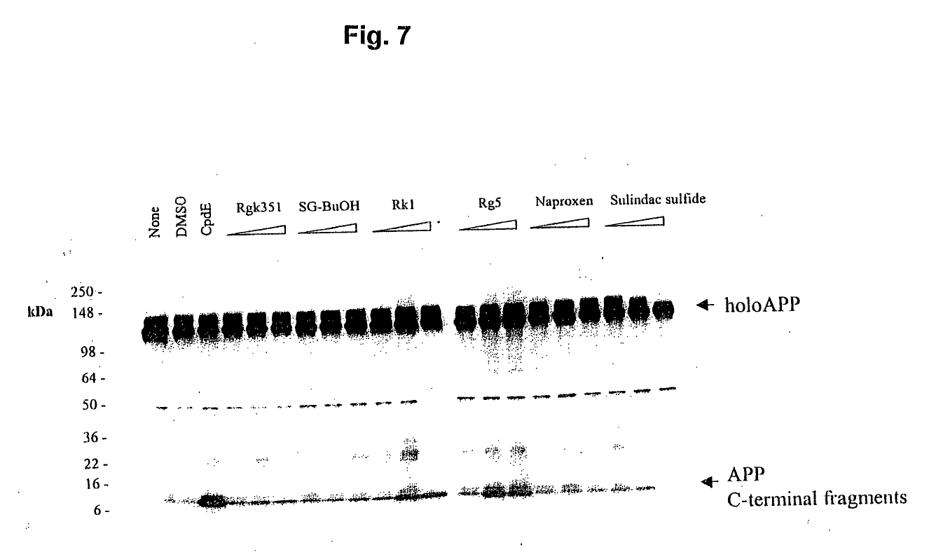

FIG. 8 demonstrates that treatment of Rgk351 and Rk1 reduced the Aβ42 levels in CHO cells co-expressing human APP together with either wild-type presenilin 1 or familial Alzheimer-linked mutant forms of presenilin 1 (delta E9 ad L286V). The effects of Rg5 on the Aβ42 generation were much smaller as compared to Rgk351 and Rk1.

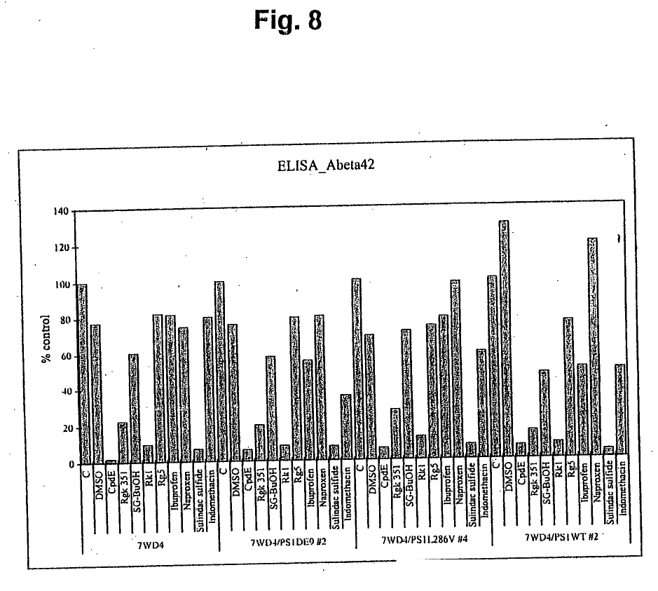

FIG. 9 shows effects of Rk1(R1) and Rg5(R5) on Aβ42-specific γ-secretase activity. Naproxen (NP) and sulindac sulfide (SS) were tested in parallel.

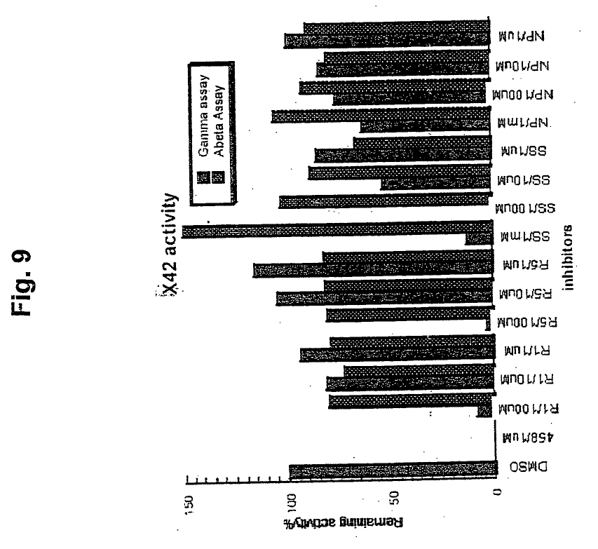

FIG. 10 depicts the effects of native ginsenosides on Aβ42 production. The structures of seven standard ginsenosides studied (Rb1, Rb2, Rc, Rd, Re, Rg1, and Rg2) are shown in Table 1. CHO cells stably transfected with human APP695 together with either wild-type (A, CHO-APP/PS1 cells) or ΔE9 FAD mutant (B, CHO-APP/ΔE9PS1 cells) forms of PS1 were used. Cells were treated with the indicated compounds (at 50 μM) for 8 hrs. Levels of secreted Aβ40 and Aβ42 in the medium were determined by ELISA and normalized to intracellular full-length APP. In CHO-APP/PS1 cells, average Aβ amounts in control samples were 320 pM for Aβ40 and 79 pM for Aβ42. The relative levels of Aβ and Aβ42 were normalized to values obtained from non-treated and vehicle-treated cells and are shown as % to control+s.d.). One of three representative experiments are shown.

FIG. 11 shows Aβ42-lowering activity of several ginsenosides derived from heat- or steam-processed ginseng. CHO-APP/PS1 (A) and CHO-APP/ΔE9PS1 (B) cells were treated with the indicated compounds at 50 μM for 8 hrs and the levels of secreted Aβ40 and Aβ42 were determined as described in FIG. 1. Note that the potency of Aβ42-reducing activity was in order of Rk1>/=(20S)Rg3>Rg5>(20R)Rg3, and the effects of Rh1 and Rg6 were not significant. Rh2 also exhibited Aβ42-lowering effects although the cell viability was partially affected at 50 μM treatment (data not shown). The PS1-ΔE9 FAD mutation diminished the Aβ42 response to Rk1 treatment (B).

FIG. 12 shows treatment with Rgk351, Rk1 and Rg5 reduced Aβ42 in the medium of CHO-APP cells in a dose-dependent manner. (A) Dose-response of Aβ42 lowering activity of Rk1 and Rg5. IC50 of Rk1 was about 20 μM. (B) Rk1 preferentially lowers Aβ42 (vs. Aβ40) in cultured CHO-APP cells and the Aβ42-inhibition pattern of Rk1 is similar to that of sulindac sulfide (SS). The relative levels of Aβ40 and Aβ42 were normalized to values obtained from non-treated and vehicle-treated cells. Similar data were obtained using Neuro2a-sw (mouse Neuro2a cells expressing Swedish familial Alzheimer's disease mutant form of APP) and 293 cells expressing human APP (data not shown). The effects of Rg5 on the Aβ42 generation were much smaller as compared to Rgk351 and Rk1.

FIG. 13. depicts an analysis of APP processing after Rk1 treatment. Steady-state levels of full-length APP and APP C-terminal fragments (APP-CTFs) were examined by Western blot analysis using anti-R1 antibody. Rgk351(mixture of Rg3, Rg5 and Rk1), Rk1 and Rg5 treatment resulted in increased accumulation of APP C-terminal fragments (γ-secretase substrates) in CHO-APP cells and mouse neuroblastoma neuro2a cells stably expressing Swedish FAD mutant form (KM670/671NL) of APP (APPsw). Correlated Aβ42 levels for each sample are shown in the bottom panel.

FIG. 14 shows that Aβ42-lowering ginsenoside Rk1 does not significantly affect the production of intracellular domains (ICDs) from APP (A, AICD), Notch1 (B, NICD) or p75 neurotrophin receptor (p75NTR, p75-ICD). Membrane fractions isolated from 293 cells overexpressing either APP (A), Notch-AE (B) or p75-AE (C) and incubated in the presence of indicated compounds: Compound E (CpdE, general γ-secretase inhibitor), Rgk351, Rk1 and sulindac sulfide (SS). Very low amounts of AICD, NICD and p75-ICD were detected in control samples (-Incubate) or in samples treated with Cpd.E, but AICD, NICD and p75-ICD were abundantly produced in samples incubated with Rgk351, Rk1 and SS.

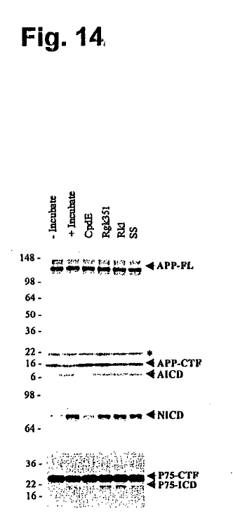

FIG. 15 shows that Aβ42-lowering ginsenoside Rk1 and (20S)Rg3 inhibits Aβ generation in a cell-free γ-secretase assay. (A) CHAPSO-solubilized membrane fractions were incubated with recombinant γ-secretase substrates together with the indicated compounds (at 100 μM) and the levels of Aβ42 and Aβ40 were determined by ELISA as described (27-29). (B) Dose-response of Aβ40 and Aβ42-lowering activity of Rk1 and (20S)Rg3 in a cell-free γ-secretase assay. IC₅₀ of Rk1 was 27±3 μM for Aβ40 and 32±5 for Aβ42. IC₅₀ of (20S)Rg3 was 27±4 for Aβ40 and 26±7 for Aβ42.

FIG. 16 depicts the effects of two major metabolites of ginsenosides, including 20(S)-protopanaxatriol (PPT) and 20(S)-protopanaxadiol (PPD) on Aβ42 generation. 20(S)-panaxatriol (PT) and 20(S)-panaxadiol (PD) are the artificial derivatives of PPT and PPPD, respectively. Treatment with either PPT or PT reduced the production of Aβ42 without affecting the levels of Aβ42 in Neuro2a cells expressing the human Swedish mutant form of APP (Neuro2a-SW, bottom panel), as well as in CHO cells expressing wild-type human APP (data not shown). PPD and PD did not confer any inhibitory effects on Aβ40 or Aβ42 generation.

FIG. 17 shows mass spectrometric analysis of Aβ species produced from CHO-APP cells treated with DMSO (vehicle), Rk1, or (20S)Rg3. Note that treatment leads to a decrease in Aβ42 species (1-42), and elevation in both Aβ37 (1-37) and Aβ38 (1-38). Mass spectrometric analysis of Aβ species were performed as previously described (Wang R, Sweeny D, Gandy S E, Sisodia S S. The profile of soluble amyloid β-protein in cultured cell media. J. Bio. Chem. 1996; 271: 31894-31902).

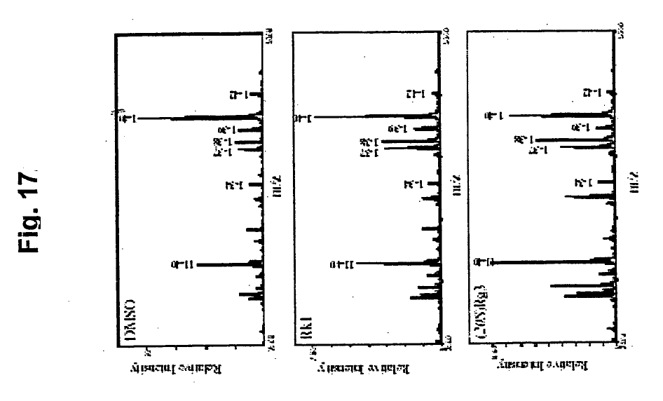

FIG. 18 depicts analysis of secreted Aβ levels after treatment of CHO-APP cells with DMSO (Control 1), naproxen (Control 2), Rk1, or (20S)Rg3. AP was immoprecipitated using 4G8 antibody (Purchased from Senetek), subjected to SDS-PAGE using Tricine/Urea gel (the protocol was supplied by Dr. Y. Ihara, University of Tokyo), and analyzed by Western blot analysis using the 6E10 antibody (Senetek). Synthetic Aβ40 and Aβ42 peptides were used to identify corresponding Aβ species.

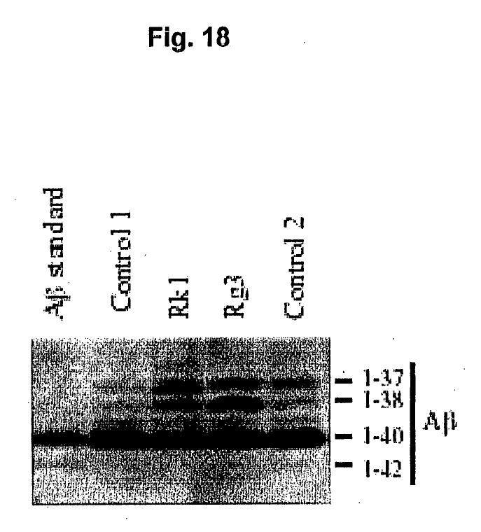

FIG. 19 shows the effects of the ginsenoside Rk1 and (20S)Rg3 on Aβ40 and Aβ42 secretion in primary embryonic cortical neurons derived from Tg2576 transgenic mice. Treatment of Rk1 and Rg3 decreased the level of secreted Aβ40 and Aβ42.

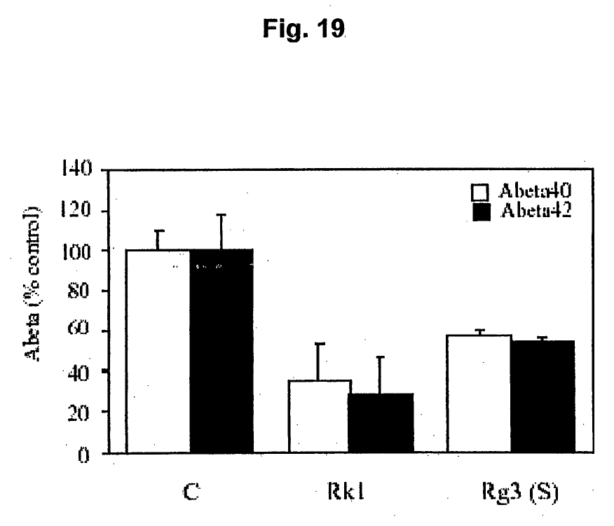

### DETAILED DESCRIPTION OF THE INVENTION

As used herein and in the appended claims, the singular forms “a,” “an,” and “the” include plural references unless the content clearly dictates otherwise. Thus, for example, reference to “an agent” includes a plurality of such agents, and reference to “the ginsenoside” is a reference to one or more ginsenodies and equivalents thereof known to those skilled in the art, and so forth. All publications, patent applications, patents, and other references mentioned herein are incorporated by reference in their entirety.

In accordance with the present invention, compounds and methods for treating Alzheimer's disease, neurodegeneration and for modulating the production of amyloid-beta protein (Aβ) are provided.

In one aspect, the present invention provides a compound having the general formula: 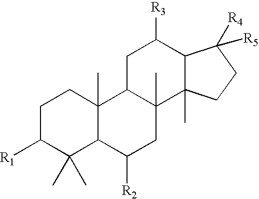
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH. The alkyl I group may further contain oxygen, nitrogen, or phosphorus and the alkyl II group may further contain a function group, such as hydroxyl, ether, ketone, oxime, hydrazone, imine, and Schiff base. In one embodiment, the sugar is selected from a group comprising Glc, Ara(pyr), Ara(fur), Rha, and Xyl. In another embodiment, R₄ is selected from the group consisting of: 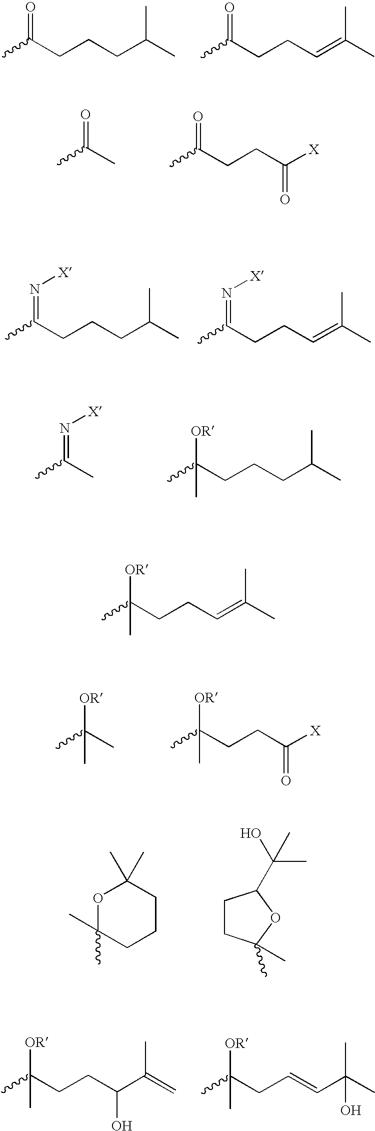
wherein the configuration of any stereo-center is R or S; X is OR or NR, wherein R is alkyl or aryl; X′ is alkyl, OR, NR, wherein R is alkyl or aryl; and R′ is H, alkyl, or acyl. As disclosed herein, the compounds are dammaranes, particularly ginsenosides and their analogues. As used herein, the term “ginsenoside” refers to the class of triterpene glycosides which includes, without limitation, the specific compounds Ra1, Ra2, Ra3, Rb1, Rb2, Rb3, Rc, Rd, Re, Rf, Rg1, (20R)Rg2, (20S)Rg2, (20R)Rg3, (20S)Rg3, Rg5, Rg6, Rh1, (20R)Rh2, (20S)Rh2, Rh3, Rh4, (20R)Rg3, (20S)Rg3, Rk1, Rk2, Rk3, Rs1, Rs2, Rs3, Rs4, Rs5, Rs6, Rs7, F4, Rgk351, protopanaxadiol (PPD), protopanaxatriol (PPT), DHPPD-I, DHPPD-II, DHPPT-I, DHPPT-II, a butanol-soluble fraction of sun ginseng, white ginseng or red ginseng or analogues or homologues thereof. The ginsenosides of the present invention may be chemically associated with carbohydrates including, but not limited to, glucopyranosyl, arabinopyranosyl, arabinofuranosyl and rhamnopyranosyl. The ginsenosides of the present invention may be isolated ginsenoside compounds or isolated and further synthesized ginsenosides. The isolated ginsenosides of the present invention can be further synthesized using processes including, but not necessarily limited to, heat, light, chemical, enzymatic or other synthesis processes generally known to the skilled artisan.

The present invention further provides a method for the synthesis of a compound having formula: 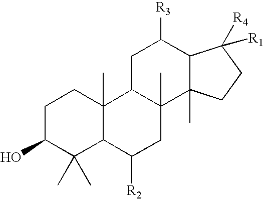
wherein the method comprises the steps of:
(a) treating a compound having formula: 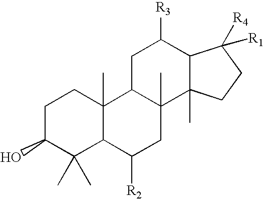
   with an oxidizing agent, to form a compound having formula: 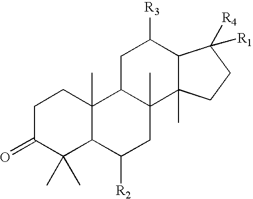
(b) treating the compound formed in step (a) with a reducing agent, to form a compound having formula: 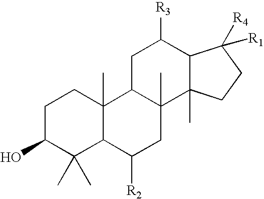
   wherein R₁ is H or OH; R₂ is selected from the group consisting of H, OH, OAc, and O—X, wherein X is a carbohydrate containing one or more sugars or acylated derivatives thereof; R₃ is selected from the group consisting of H, OH, and OAc; and R₄ is alkenyl, aryl, or alkyl. In one embodiment, the oxidizing agent is chromic anhydride and the reducing agent is NaBH₄.

The starting material, i.e. the compound having formula: 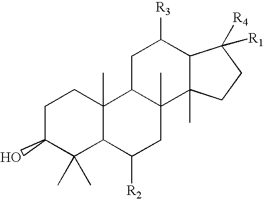
particularly, betulafolienetriol, may be obtained from plants including, without limitation, common birch. The extracts of these plants are rich sources of betulafolienetriol and are desired starting materials for making ginsenosides because they cost significantly less than ginseng.

The present invention also provides a method for the synthesis of a compound having formula: 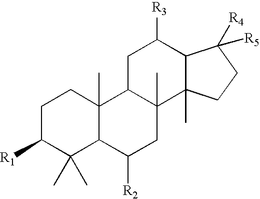
wherein the method comprises the steps of:
(a) treating a compound having formula: 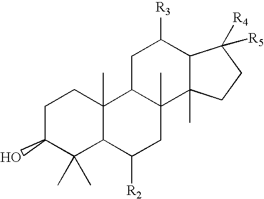
   with an oxidizing agent, to form a compound having formula: 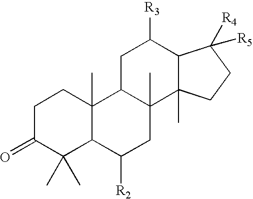
(b) treating the compound formed in step (a) with a reducing agent, to form a compound having formula: 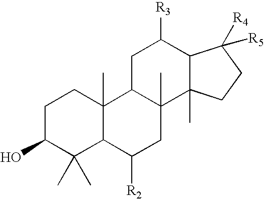
(c) optionally, treating the compound formed in step (b) with protected R₁ derivative, to form a compound having formula: 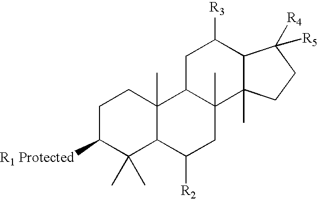
(d) treating the compound formed in step (c) with deprotection agent, to form a compound having formula: 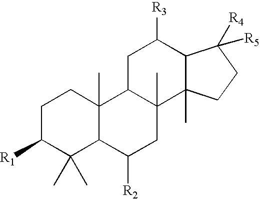
   wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof, R₃ is selected from the group consisting of H, OH, and OAc; R₄ is alkenyl, aryl, or alkyl II; and R₅ is H or OH. The alkyl I group may further contain oxygen, nitrogen, or phosphorus; and the alkyl II group may further contain a function group, such as hydroxyl, ether, ketone, oxime, hydrazone, imine, and Schiff base. In one embodiment, the oxidizing agent is chromic anhydride and the reducing agent is NaBH₄. In another embodiment, the protected R₁ derivative is a protected R₁ halogen derivative. For example, the protected R₁ derivative may be protected by an Ac₈-group. The protected R₁ group may be deprotected using agents such as NaOMe.

Additionally, the present invention provides a method for the synthesis of a compound having formula: 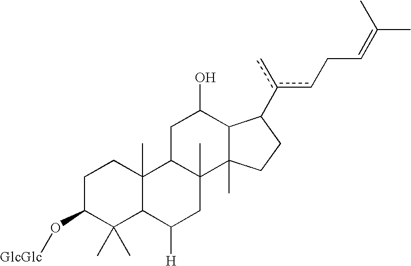
wherein the method comprises the steps of:
(a) treating a compound having formula: 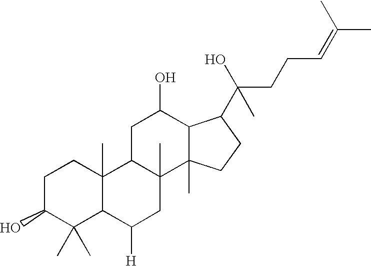
   with an oxidizing agent, to form a compound having formula: 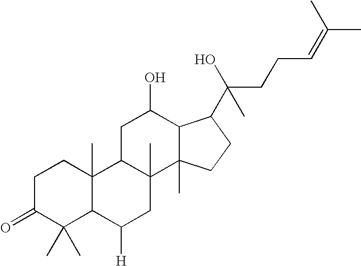
(b) treating the compound formed in step (a) with a protecting agent, to form a compound having formula: 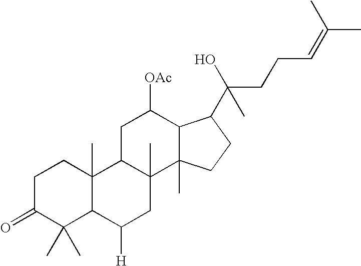
(c) treating the compound formed in step (b) with a reducing agent, to form a compound having formula: 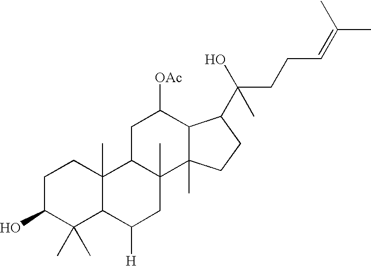
(d) treating the compound formed in step (c) with Ac₈-Glc-Glc-Br, to form a compound having formula: 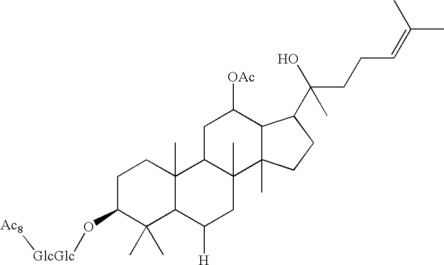
(e) treating the compound formed in step (d) with deprotection agent, to form a compound having formula: 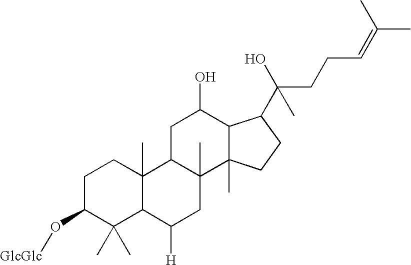
(f) further modifying the compound formed in step (e) to form a compound having formula: 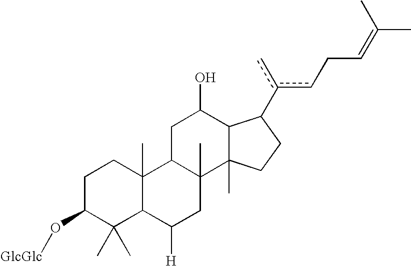
   In one embodiment, the oxidizing agent is chromic anhydride, the reducing agent is NaBH₄, the compound is deprotected using NaOMe.

The present invention also provides a method for the synthesis of a compound having formula: 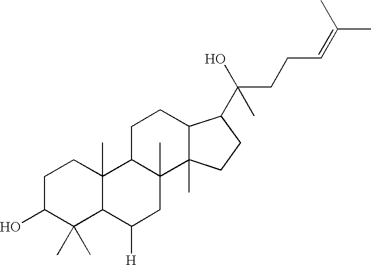
wherein the method comprises the step of treating a compound having formula: 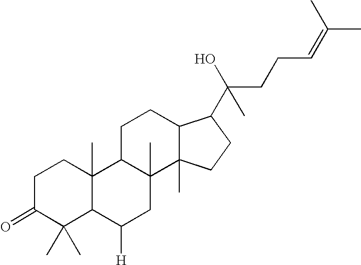
with a reducing agent, such as, NaBH₄.

Also provided is a method for the synthesis of a compound having formula: 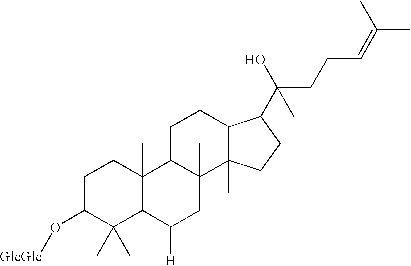
wherein the method comprises the steps of:
(a) treating a compound having formula: 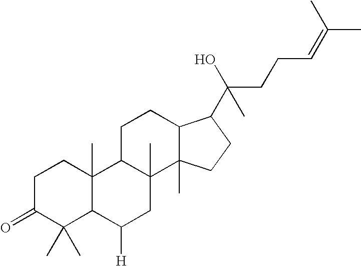
   with a reducing agent, to form a compound having formula: 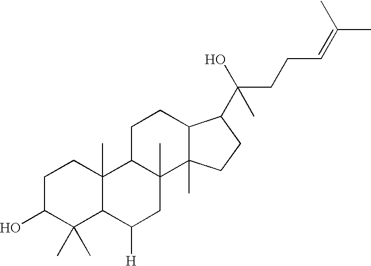
(b) treating the compound formed in step (a) with Ac₈-Glc-Glc-Br, to form a compound having formula: 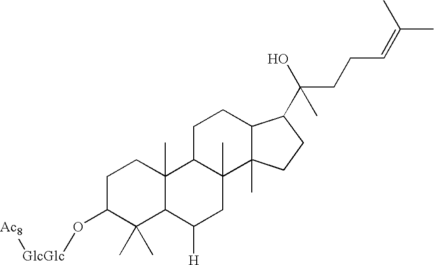
(c) treating the compound formed in step (d) with deprotection agent, to form a compound having formula: 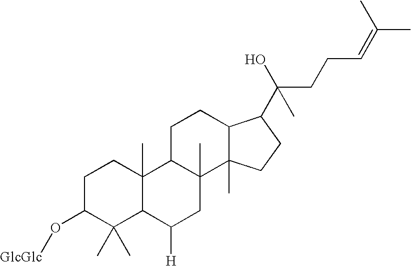
   In one embodiment, the reducing agent is NaBH₄ and the compound is deprotected using NaOMe.

Additionally, the present invention provides ginsenoside compositions for use in modulating amyloid-beta production in a subject, treating or preventing Alzheimer's disease and treating or preventing neurodegeneration comprising a mixture of isolated or isolated and further synthesized ginsenosides, wherein one or more of the ginsenosides is selected from the group consisting of: Ra1, Ra2, Ra3, Rb1, Rb2, Rb3, Rc, Rd, Re, Rf, Rg1, (20R)Rg2, (20S)Rg2, (20R)Rg3, (20S)Rg3, Rg5, Rg6, Rh1, (20R)Rh2, (20S)Rh2, Rh3, Rh4, (20R)Rg3, (20S)Rg3, Rk1, Rk2, Rk3, Rs1, Rs2, Rs3, Rs4, Rs5, Rs6, Rs7, F4, protopanaxadiol (PPD), protopanaxatriol (PPT), DHPPD-I, DHPPD-II, DHPPT-I, DHPPT-II, a butanol-soluble fraction of sun ginseng, white ginseng or red ginseng or analogues or homologues thereof. In an embodiment of the invention, the ginsenoside composition is Rgk351.

The present invention provides methods and pharmaceutical compositions for use in decreasing amyloid-beta production, comprising use of a pharmaceutically-acceptable carrier and a ginsenoside compound. Examples of acceptable pharmaceutical carriers, formulations of the pharmaceutical compositions, and methods of preparing the formulations are described herein. The pharmaceutical compositions may be useful for administering the dammarane and ginsenoside compounds of the present invention to a subject to treat a variety of disorders, including neurodegeneration and/or its associated symptomology, as disclosed herein. The ginsenoside compound is provided in an amount that is effective to treat the disorder (e.g., neurodegeneration) in a subject to whom the pharmaceutical composition is administered. The skilled artisan, as described above, may readily determine this amount. In one embodiment, the present invention provides a method for inhibiting β-amyloid production in a subject, comprising administering a compound having the general formula: 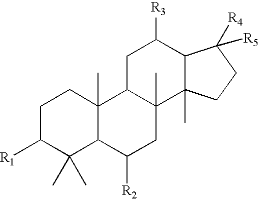
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH. As used herein, the term “subject” includes, for example, an animal, e.g. human, rat, mouse, rabbit, dog, sheep, and cow, as well as an in vitro system, e.g. a cultured cell, tissue, and organ.

The present invention also provides a method for treating neurodegeneration in a subject in need of treatment, by contacting cells (preferably, cells of the CNS) in the subject with an amount of a ginsenoside compound or composition effective to decrease amyloid-beta production in the cells, thereby treating the neurodegeneration. Examples of neurodegeneration which may be treated by the method of the present invention include, without limitation, Alzheimer's disease, amyotrophic lateral sclerosis (Lou Gehrig's disease), Binswanger's disease, corticobasal degeneration (CBD), dementia lacking distinctive histopathology (DLDH), frontotemporal dementia (FTD), Huntington's chorea, multiple sclerosis, myasthenia gravis, Parkinson's disease, Pick's disease, and progressive supranuclear palsy (PSP). In a preferred embodiment of the present invention, the neurodegeneration is Alzheimer's disease (AD) or sporadic Alzheimer's disease (SAD). In a further embodiment of the present invention, the Alzheimer's disease is early-onset familial Alzheimer's disease (FAD). The skilled artisan can readily determine when clinical symptoms of neurodegeneration have been ameliorated or minimized.

The present invention also provides a method for treating or preventing a pathological condition, such as neurodegeneration and Aβ42-related disorder, in a subject in need of treatment, comprising administering to the subject one or more ginsenoside compounds in an amount effective to treat the neurodegeneration. The Aβ42-related disorder may be any disorder caused by Aβ42 or has a symptom of aberrant Aβ42 accumulation. As used herein, the phrase “effective to treat the neurodegeneration” means effective to ameliorate or minimize the clinical impairment or symptoms of the neurodegeneration. For example, where the neurodegeneration is Alzheimer's disease, the clinical impairment or symptoms of the neurodegeneration may be ameliorated or minimized by reducing the production of amyloid-beta and the development of senile plaques and neurofibrillary tangles, thereby minimizing or attenuating the progressive loss of cognitive function. The amount of inhibitor effective to treat neurodegeneration in a subject in need of treatment will vary depending upon the particular factors of each case, including the type of neurodegeneration, the stage of the neurodegeneration, the subject's weight, the severity of the subject's condition, and the method of administration. This amount can be readily determined by the skilled artisan. In one embodiment, the present invention provides a method for treating or preventing neurodegeneration in a subject, comprising administering a compound having the general formula: 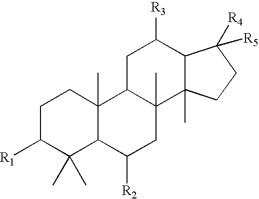
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

In one embodiment of the invention, Alzheimer's disease is treated in a subject in need of treatment by administering to the subject a therapeutically effective amount of a ginsenoside composition, a ginsenoside or analogue or homologue thereof effective to treat the Alzheimer's disease. The subject is preferably a mammal (e.g., humans, domestic animals, and commercial animals, including cows, dogs, monkeys, mice, pigs, and rats), and is most preferably a human. The term analogue as used in the present invention refers to a chemical compound that is structurally similar to another and may be theoretically derivable from it, but differs slightly in composition. For example, an analogue of the ginsesnoside (20S)Rg3 is a compound that differs slightly from (20S)Rg3 (e.g., as in the replacement of one atom by an atom of a different element or in the presence of a particular functional group), and may be derivable from (20S)Rg3. The term homologue as used in the present invention refers to members of a series of compounds in which each member differs from the next member by a constant chemical unit. The term synthesize as used in the present invention refers to formation of a particular chemical compound from its constituent parts using synthesis processes known in the art. Such synthesis processes include, for example, the use of light, heat, chemical, enzymatic or other means to form particular chemical composition.

The term “therapeutically effective amount” or “effective amount,” as used herein, means the quantity of the composition according to the invention which is necessary to prevent, cure, ameliorate or at least minimize the clinical impairment, symptoms or complications associated with Alzheimer's disease in either a single or multiple dose. The amount of ginsenoside effective to treat Alzheimer's disease will vary depending on the particular factors of each case, including the stage or severity of Alzheimer's disease, the subject's weight, the subject's condition and the method of administration. The skilled artisan can readily determine these amounts. For example, the clinical impairment or symptoms of Alzheimer's disease may be ameliorated or minimized by diminishing any dementia or other discomfort suffered by the subject; by extending the survival of the subject beyond that which would otherwise be expected in the absence of such treatment; or by inhibiting or preventing the progression of the Alzheimer's disease.

Treating Alzheimer's disease, as used herein, refers to treating any one or more of the conditions underlying Alzheimer's disease including, without limitation, neurodegeneration, senile plaques, neurofibrillary tangles, neurotransmitter deficits, dementia, and senility. As used herein, preventing Alzheimer's disease includes preventing the initiation of Alzheimer's disease, delaying the initiation of Alzheimer's disease, preventing the progression or advancement of Alzheimer's disease, slowing the progression or advancement of Alzheimer's disease, and delaying the progression or advancement of Alzheimer's disease.

Prior to the present invention, the effect of dammaranes and ginsenosides on production of beta amyloid protein was unknown. The present invention establishes that ginsenosides such as (20S)Rg3, Rk1 and Rg5 or their analogues or homologues can also be used to prevent and treat Alzheimer's disease patients. This new therapy provides a unique strategy to treat and prevent neurodegeneration and dementia associated with Alzheimer's disease by modulating the production of Aβ42. Further, neurodegeneration and dementias not associated with Alzheimer's disease can also be treated or prevented using the ginsenosides of the present invention to modulate the production of Aβ42.

The ginsenosides of the present invention include natural or synthetic functional variants, which have ginsenoside biological activity, as well as fragments of ginsenoside having ginsenoside biological activity. As further used herein, the term “ginsenoside biological activity” refers to activity that modulates the generation of the highly amyloidogenic Aβ42, the 42-amino acid isoform of amyloid β-peptide. In an embodiment of the invention, the ginsenoside reduces the generation of Aβ42 in the cells of a subject. Commonly known ginsenosides and ginsenoside compositions include, but are not limited to, Ra1, Ra2, Ra3, Rb1, Rb2, Rb3, Rc, Rd, Re, Rf, Rg1, (20R)Rg2, (20S)Rg2, (20R)Rg3, (20S)Rg3, Rg5, Rg6, Rh1, (20R)Rh2, (20S)Rh2, Rh3, Rh4, (20R)Rg3, (20S)Rg3, Rk1, Rk2, Rk3, Rs1, Rs2, Rs3, Rs4, Rs5, Rs6, Rs7, F4, Rgk351, protopanaxadiol (PPD), protopanaxatriol (PPT), DHPPD-I, DHPPD-II, DHPPT-I, DHPPT-II, a butanol-soluble fraction of sun ginseng, white ginseng or red ginseng or analogues or homologues thereof. In one embodiment of the invention the ginsenoside is Rk1. In another embodiment of the invention, the ginsenoside is (20S)Rg3. In a further embodiment, the ginsenoside is Rg5. In still another embodiment, the ginsenoside composition is Rgk351, a mixture of (20S)Rg3, Rg5 and Rk1.

Methods of preparing ginsenosides such as Rk1, (20S)Rg3 and Rg5, as well as their analogues and homologues, are well known in the art. For example, U.S. Pat. No. 5,776,460, the disclosure of which is incorporated herein in its entirety, describes preparing a processed ginseng product in which a ratio of ginsenoside (Rg3+Rg5) to (Rc+Rd+Rb1+Rb2) is above 1.0. The processed product disclosed in U.S. Pat. No. 5,776,460 is prepared by heat-treating ginseng at a high temperature of 120° to 180° C. for 0.5 to 20 hours. The ginsenosides of the present invention may be isolated ginsenoside compounds or isolated and further synthesized ginsenoside compounds. The isolated ginsenosides of the present invention can be further synthesized using processes including, but not necessarily limited to, heat, light, chemical, enzymatic or other synthesis processes generally known to the skilled artisan.

In a method of the present invention, the ginsenoside compound is administered to a subject in combination with one or more different ginsenoside compounds. Administration of a ginsenoside compound “in combination with” one or more different ginsenoside compounds refers to co-administration of the therapeutic agents. Co-administration may occur concurrently, sequentially, or alternately. Concurrent co-administration refers to administration of the different ginsenoside compounds at essentially the same time. For concurrent co-administration, the courses of treatment with the two or more different ginsenosides may be run simultaneously. For example, a single, combined formulation, containing both an amount of a particular ginsenoside compound and an amount of a second different ginsenoside compound in physical association with one another, may be administered to the subject. The single, combined formulation may consist of an oral formulation, containing amounts of both ginsenoside compounds, which may be orally administered to the subject, or a liquid mixture, containing amounts of both the ginsenoside compounds, which may be injected into the subject.

It is also within the confines of the present invention that an amount of one particular ginsenoside compound and an amount one or more different ginsenoside compound may be administered concurrently to a subject, in separate, individual formulations. Accordingly, the method of the present invention is not limited to concurrent co-administration of the different ginsenoside compounds in physical association with one another.

In the method of the present invention, the ginsenoside compounds also may be co-administered to a subject in separate, individual formulations that are spaced out over a period of time, so as to obtain the maximum efficacy of the combination. Administration of each therapeutic agent may range in duration from a brief, rapid administration to a continuous perfusion. When spaced out over a period of time, co-administration of the ginsenoside compounds may be sequential or alternate. For sequential co-administration, one of the therapeutic agents is separately administered, followed by the other. For example, a full course of treatment with an Rg5 derivative may be completed, and then may be followed by a full course of treatment with an Rk1 derivative. Alternatively, for sequential co-administration, a full course of treatment with Rk1 derivative may be completed, then followed by a full course of treatment with an Rg5 derivative. For alternate co-administration, partial courses of treatment with the Rk1 derivative may be alternated with partial courses of treatment with the Rg5 derivative, until a full treatment of each therapeutic agent has been administered.

The therapeutic agents of the present invention (i.e., the ginsenoside and analogues and analogues thereof) may be administered to a human or animal subject by known procedures including, but not limited to, oral administration, parenteral administration (e.g., intramuscular, intraperitoneal, intravascular, intravenous, or subcutaneous administration), and transdermal administration. Preferably, the therapeutic agents of the present invention are administered orally or intravenously.

For oral administration, the formulations of the ginsenoside may be presented as capsules, tablets, powders, granules, or as a suspension. The formulations may have conventional additives, such as lactose, mannitol, corn starch, or potato starch. The formulations also may be presented with binders, such as crystalline cellulose, cellulose analogues, acacia, cornstarch, or gelatins. Additionally, the formulations may be presented with disintegrators, such as cornstarch, potato starch, or sodium carboxymethyl cellulose. The formulations also may be presented with dibasic calcium phosphate anhydrous or sodium starch glycolate. Finally, the formulations may be presented with lubricants, such as talc or magnesium stearate.

For parenteral administration, the formulations of the ginsenoside may be combined with a sterile aqueous solution which is preferably isotonic with the blood of the subject. Such formulations may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampules or vials. Moreover, the formulations may be delivered by any mode of injection including, without limitation, epifascial, intracapsular, intracutaneous, intramuscular, intraorbital, intraperitoneal (particularly in the case of localized regional therapies), intraspinal, intrasternal, intravascular, intravenous, parenchymatous, or subcutaneous.

For transdermal administration, the formulations of the ginsenoside may be combined with skin penetration enhancers, such as propylene glycol, polyethylene glycol, isopropanol, ethanol, oleic acid, N-methylpyrrolidone, and the like, which increase the permeability of the skin to the therapeutic agent, and permit the therapeutic agent to penetrate through the skin and into the bloodstream. The therapeutic agent/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in a solvent such as methylene chloride, evaporated to the desired viscosity, and then applied to backing material to provide a patch.

The dose of the ginsenoside of the present invention may also be released or delivered from an osmotic mini-pump. The release rate from an elementary osmotic mini-pump may be modulated with a microporous, fast-response gel disposed in the release orifice. An osmotic mini-pump would be useful for controlling release, or targeting delivery, of the therapeutic agents.

It is within the confines of the present invention that the formulations of the ginsenoside may be further associated with a pharmaceutically-acceptable carrier, thereby comprising a pharmaceutical composition. The pharmaceutically-acceptable carrier must be “acceptable” in the sense of being compatible with the other ingredients of the composition, and not deleterious to the recipient thereof. Examples of acceptable pharmaceutical carriers include, but are not limited to, carboxymethyl cellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may conveniently be presented in unit dosage.

The formulations of the present invention may be prepared by methods well known in the pharmaceutical art. For example, the active compound may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the therapeutic agents of the present invention (i.e., ginsenosides their analogues and analogues, either in separate, individual formulations, or in a single, combined formulation) to a subject to treat Alzheimer's disease. The therapeutic agents are provided in amounts that are effective to treat or prevent Alzheimer's disease in the subject. These amounts may be readily determined by the skilled artisan.

The effective therapeutic amounts of the ginsenoside will vary depending on the particular factors of each case, including the stage of the Alzheimer's disease, the subject's weight, the severity of the subject's condition, and the method of administration. For example, (20S)Rg3 can be administered in a dosage of about 5 μg/day to 1500 mg/day. Preferably, (20S)Rg3 is administered in a dosage of about 1 mg/day to 1000 mg/day. Rg5 can be administered in a dosage of about 5 μg/day to 1500 mg/day, but is preferably administered in a dosage of about 1 mg/day to 1000 mg/day. Rk1 can be administered in a dosage of about 5 μg/day to 1500 mg/day, but is preferably administered in a dosage of about 1 mg/day to 1000 mg/day. Further, the ginsenoside composition Rgk351 can be administered in a dosage of about 5 μg/day to 1500 mg/day, but is preferably administered in a dosage of about 1 mg/day to 1000 mg/day. The appropriate effective therapeutic amounts of any particular ginsenoside compound within the listed ranges can be readily determined by the skilled artisan depending on the particular factors of each case.

The present invention additionally encompasses methods for preventing Alzheimer's disease in a subject with a pre-Alzheimer's disease condition, comprising administering to the subject a therapeutically effective amount of a ginsenoside compound. As used herein, “pre-Alzheimer's disease condition” refers to a condition prior to Alzheimer's disease. The subject with a pre-Alzheimer's disease condition has not been diagnosed as having Alzheimer's disease, but nevertheless may exhibit some of the typical symptoms of Alzheimer's disease and/or have a medical history likely to increase the subject's risk to developing Alzheimer's disease.

The invention further provides methods for treating or preventing Alzheimer's disease in a subject, comprising administering to the subject a therapeutically effective amount of ginsenoside compound.

### EXAMPLES

The following examples illustrate the present invention, which are set forth to aid in the understanding of the invention, and should not be construed to limit in any way the scope of the invention as defined in the claims which follow thereafter.

The inventors have unexpectedly found that at least three Ginsenoside compounds, Rk1, (20S)Rg3 and Rg5 as well as the mixture Rgk351, lower the production of Aβ42 in cells, thus treating AD and non-AD associated neuropathogenesis and/or preventing the progression of AD and non-AD associated neuropathogenesis. Rgk351 and Rk1 were most effective in reducing Aβ42 levels. Further, Rk1 was shown to inhibit the Aβ42 production in the cell-free assay using a partially purified γ-secretase complex, suggesting that Rk1 modulates either specificity and/or activity of the γ-secretase enzyme.

### Example 1

The potential effects of ginsenosides and their analogues in treating AD were examined. First, a number of ginsenosides were screened based on their effects on Aβ generation. The effects of various ginsenosides on Aβ (e.g., Aβ40 and Aβ42) production was initially accessed by incubating the Chinese hamster ovary (CHO) cells expressing human APP(CHO-APP cells) with each ginsenoside purified from unprocessed ginseng (known as “white ginseng”). These representative ginsenosides included Rb1, Rb2, Rc, Rd, Re, Re, Rg1 and Rg2 and differ in their side chains and sugar moieties.

Tables 1-3 Structure of ginsenosides utilized in the study and their effects on Aβ42 generation. They differ at the two or three side chains attached to the common triterpene backbone known as dammarane. The common structure skeleton for each group of ginsenosides is shown in the top panel. Ginsenosides that harbor Aβ42-lowering activity are indicated in the far right column of the tables: Aβ42-lowering activity (“Yes”), no profound effects (“No”), and non-determined (“ND”). Ginsenosides that affected cell viability are indicated as “Cytotoxic.” Abbreviation for carbohydrates are as follows: Glc, D-glucopyranosyl; Ara (pyr), L-arabinopyranosyl; Ara (fur), L-arabinofuranyosyl; Rha, L-rhamnopyranosyl.

TABLE 1

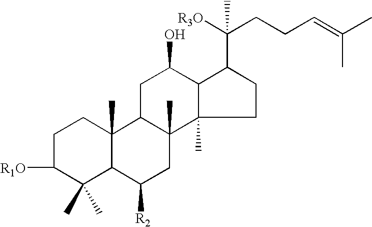

| | | | | Aβ42-lowering |
|---|---|---|---|---|
| Ginsenoside | R1 | R2 | R3 | activity |
| PPD (Protopanaxadiol) | —H | —H | —H | No |
| Ra1 | -Glc-Glc | —H | -Glc-Ara (pyr)-Xyl | ND |
| Ra2 | -Glc-Glc | —H | -Glc-Ara (fur)-Xyl | ND |
| Ra3 | -Glc-Glc | —H | -Glc-Glc-Xyl | ND |
| Rb1 | -Glc-Glc | —H | -Glc-Glc | No |
| Rb2 | -Glc-Glc | —H | -Glc-Ara (pyr) | No |
| Rb3 | -Glc-Glc | —H | -Glc-Xyl | No |
| Rc | -Glc-Glc-AC | —H | -Glc-Ara (fur) | No |
| Rd | -Glc-Glc-AC | —H | -Glc | No |
| Rg3 (20R) | -Glc-Glc-AC | —H | —H | Yes |
| Rg3 (208) | -Glc-Glc | —H | —H | Yes |
| Rh2 (20R,S) | -Glc | —H | —H | Yes/Cytotoxic |
| Rs1 | -Glc-Glc | —H | -Glc-Ara (pyr) | ND |
| Rs2 | -Glc-Glc | —H | -Glc-Ara (fur) | ND |
| Rs3 | -Glc-Glc | —H | —H | Yes/Cytotoxic |
| PPT (Protopanaxatiol) | —H | —OH | —H | Yes |
| Re | —H | —O-Glc- | -Glc | No |
| Rf | —H | Rha | —H | ND |
| Rg1 | —H | —O-Glc- | -Glc | No |
| | | Glc | | |
| Rg2 (20R,S) | —H | —O-Glc | —H | No |
| Rh1 (20R,S) | —H | —O-Glc- | —H | No |
| | | Rha | | |
| | | —O-Glc | | |

TABLE 2

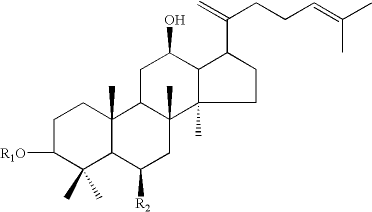

| | | | Aβ42-lowering |
|---|---|---|---|
| Ginsenoside | R1 | R2 | activity |
| DHPPD-I | H | H | ND |
| (Double-bond PPD) | | | |
| Rk1 | -Glc-Glc | —H | Yes |
| Rk2 | -Glc | —H | ND |
| Rs5 | -Glc-Glc-Ac | —H | Yes/Cytotoxic |
| DHPPT-I | —H | —OH | ND |
| (Double-bond PPT) | | | |
| Rg6 | —H | —O-Glc-Rha | No |
| Rk3 | —H | —O-Glc | No |
| Rs7 | —H | —O-Glc-Ac | ND |

TABLE 3

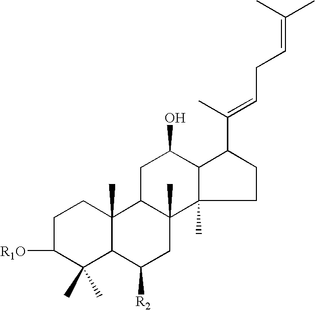

| Ginsenoside | R1 | R2 | Aβ42-lowering activity |
|---|---|---|---|
| DHPPD-II | H | —H | ND |
| Rg5 | -Glc-Glc | —H | Yes |
| Rh3 | -Glc | —H | ND |
| Rs4 | -Glc-Glc-Ac | —H | ND |
| DHPPT-II | —H | —OH | ND |
| F4 | —H | —O-Glc-Rha | ND |
| Rh4 | —H | —O-Glc | No |
| Rs6 | —H | —O-Glc-Ac | ND |

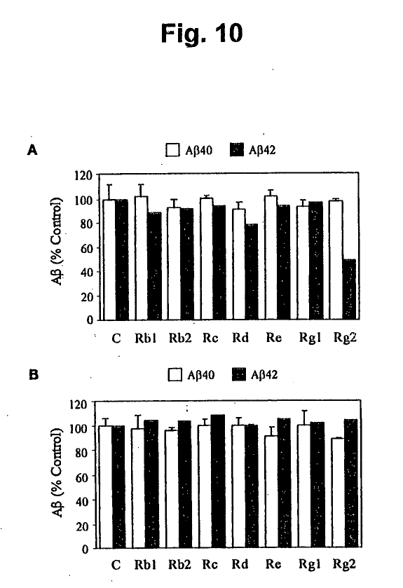

After 8 hours of incubation, the media were collected and the levels of secreted Aβ40 and Aβ42 were determined by ELISA. None of the ginsenosides from the group Rb1, Rb2, Rc, Rd, Re, Re, Rg1 and Rg2 exhibited any inhibitory effects on Aβ40 and Aβ42 production (FIG. 10).

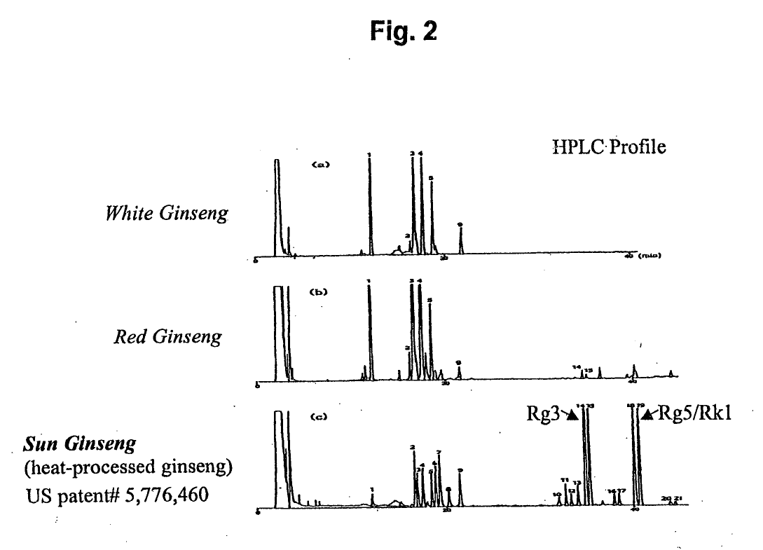

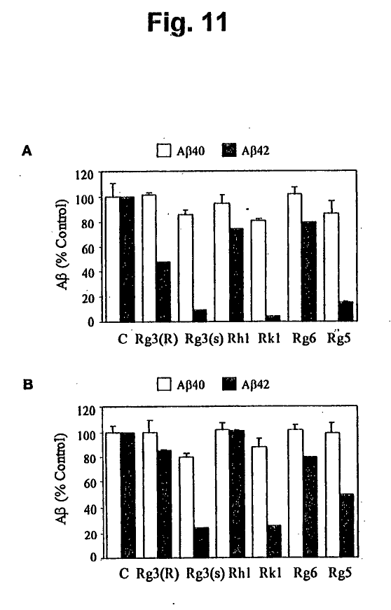

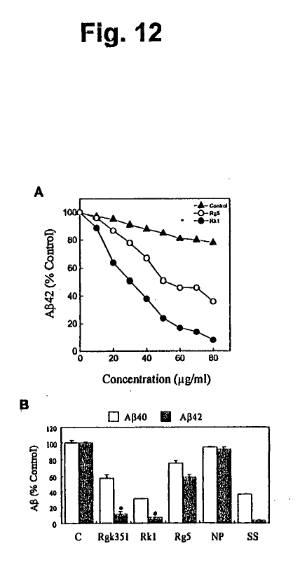

Steaming ginseng at high temperature gave rise to additional ginsenosides with enhanced pharmacological activity, including (20S)Rg3, Rk1 and Rg5 (22-25). Next, the effects of these heat-processing derived ginsenosides (e.g., (20S)Rg3, Rh1, Rh2, Rk1, Rg6, Rg5) on Aβ40 and Aβ42 generation were tested. Initial screening identified three structurally related ginsenosides, Rk1, (20S)Rg3, and Rg5, which selectively lowered the secretion of Aβ42 (FIG. 11). In contrast, Aβ42 levels were not affected by (20R)Rg3, Rh1, and Rg6. Aβ40 levels were not changed by treatment with any of the ginsenosides tested. The potency of Aβ42-lowering activity was highest with Rk1 and (20S)Rg3. Rg5 was a less effective Aβ42-lowering reagent as compared to Rk1 or (20S)Rg3 (FIG. 2). The secretion of Aβ40 was affected by treatment with Rk1 only at very high concentration (˜100 μM) and cell viability was not affected by treatment of Rk1 under these conditions (up to 100 μM, 8 hour treatment; data not shown). Interestingly, the PS1 ΔE9 FAD mutation diminished Aβ42-lowering response to (20S)Rg3, Rk1 and Rg5 treatment (FIG. 11B) as compared to PS1 wild-type expressing cells (FIG. 11A). Further analyses revealed that Rk1 and Rg5 lower Aβ42 in a dose-dependent manner (FIG. 12A). Overnight treatment with Rgk351, Rk1, and Rg5 also reduce Aβ42 production in CHO-APP cells (FIG. 12B). Aβ42-lowering activity of Rk1 was similar to that of sulindac sulfide, one of the known Aβ42-lowering NSAIDs. During overnight treatment, Aβ40 production was also slightly affected by treatment with Rk1 or sulindac sulfide (FIG. 12B). These studies provide a structure-activity relationship between the chemical structures of ginsenosides and Aβ42-lowering activity, further providing the basis for designing additional Aβ42-lowering analogues as well as for defining a class of compounds that harbor Aβ42-lowering activity.

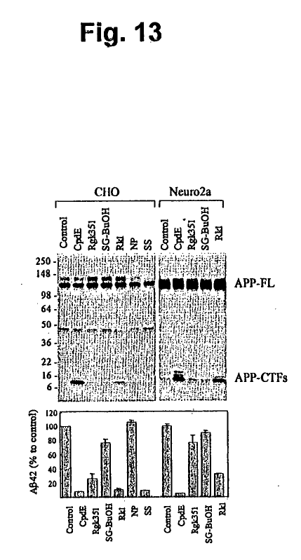

Rk1 did not affect steady-state levels of full-length APP in both CHO-APP and Neuro2a-APPsw cells (FIG. 13), suggesting that the reduction of Aβ42 is likely due to altered post-translation processing of APP. In contrast to the full-length form, the steady-state levels of C-terminal APP fragments were up-regulated by treatment with Rk1 (FIG. 13). These data suggest that Rk1 may affect the g-secretase cleavage step (e.g., Aβ42 cleavage), therefore causing the accumulation of APP C-terminal fragments, as has been shown for a general γ-secretase inhibitor Compound E. Aβ42 levels in the medium of each corresponding samples are shown in the bottom panel.

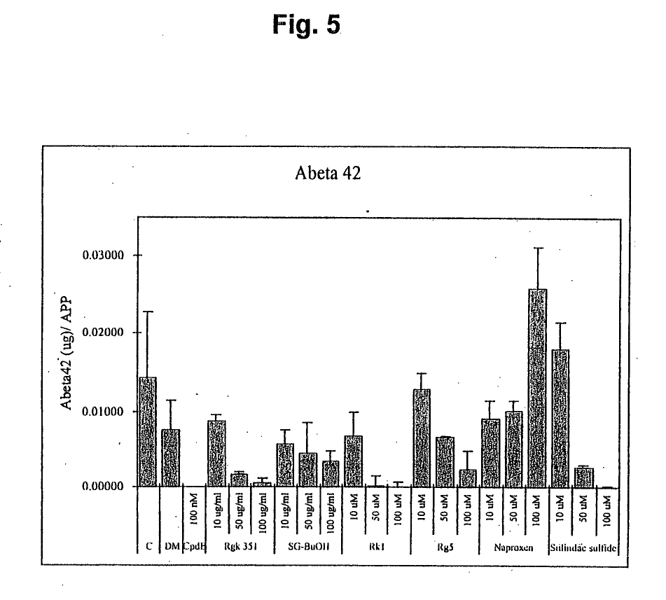

Since the effect of Rk1 was rather selective to Aβ42 (but not Aβ40) in a cell-based assay, the question of whether Rk1 affects other γ-secretase-mediated cleavage events, including the generation of AICD resulted from a transmembrane cleavage of APP distal from either Aβ40 or Aβ42 site, and γ-secretase-mediated intramembrane cleavage of Notch1 or p75 neurotrophin receptor (p75NTR) to yield Notch1 or p75NTR intracellular domains (NICD or p75-ICD, respectively) was tested. The cell-free generation of AICD, NICD and p75-ICD was not affected by incubation with Rgk351 or Rk1 (FIG. 5). Under these conditions, Compound E efficiently inhibited the cell-free generation of ICDs and sulinac sulfide did not affect ICD generation from APP, Notch1 or p75NTR. These data indicate that Rk1 is not a general inhibitor of γ-secretase cleavage and does not affect the intramembrane cleavage of other γ-secretase substrate, such as Notch1 or p75NTR.

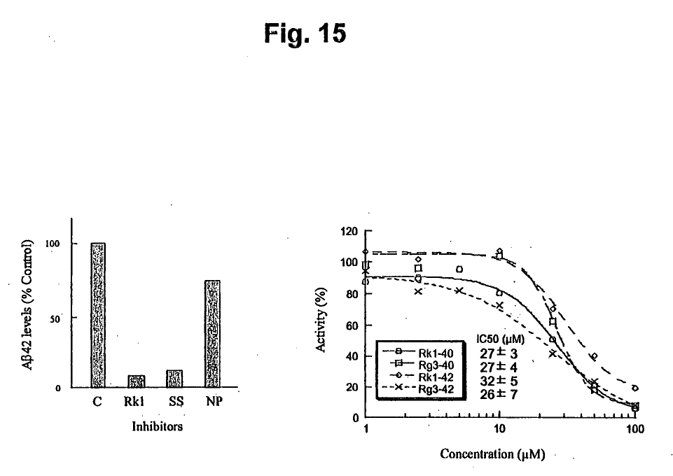

Next, the inhibitory effects of Rk1 and (20S)Rg3 on Aβ generation in an in vitro γ-secretase assay was studied. Both Rk1 and sulindac sulfide potently inhibited Aβ42 generation in vitro (FIG. 15). In contrast, naproxen, an NSAID without Aβ42-lowering activity, had no effects on Aβ42 production (FIG. 15A). Similar to what has been reported for Aβ42-lowering NSAIDs (Weggen, et al., Evidence that nonsteroidal anti-inflammatory drugs decrease amyloid beta 42 production by direct modulation of gamma-secretase activity, J. Biol. Chem. 278:3183-3187 (2003)), Aβ42-lowering ginsenosides (e.g., Rk1 and (20S)Rg3) inhibited both Aβ40 and Aβ42 with a similar potency in a cell-free γ-secretase assay (FIG. 15B), although both compounds primarily affect Aβ42 production in cell-based assay.

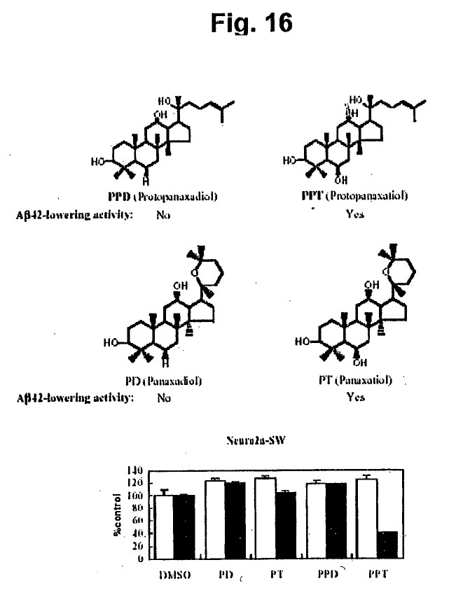

Ginsenosides are metabolized by human intestinal bacteria after oral administration of ginseng extract (Kobayashi K., et al., Metabolism of ginsenoside by human intestinal bacteria [II] Ginseng Review 1994; 18: 10-14; Hasegawa H., et al., Main ginseng saponin metabolites formed by intestinal bacteria. Planta Med. 1996; 62: 453-457.). Therefore, the effects of two major metabolites of ginsenosides, including 20(S)-protopanaxatriol (PPT) and 20(S)-protopanaxadiol (PPD) on Aβ42 generation were tested. 20(S)-panaxatriol (PT) and 20(S)-panaxadiol (PD) are the artificial derivatives of PPT and PPPD, respectively. Treatment with either PPT or PT reduced the production of Aβ42 without affecting the levels of Aβ42 in Neuro2a cells expressing the human Swedish mutant form of APP (Neuro2a-SW) as well as in CHO cells expressing wild-type human APP (FIG. 16). PPD and PD did not confer any inhibitory effects on Aβ40 or Aβ42 generation.

In summary, Aβ42-lowering natural compounds that originate from heat-processed ginseng have been identified. Aβ42-lowering ginsenosides, including Rk1 and (20S)Rg3, appear to specifically modulate γ-secretase activity that is involved in Aβ42 production. Structure-activity defines a class of compounds that could serve as a foundation for development of effective therapeutic agents for treatment of AD.

### Example 2

The benefits of ginsenoside therapy for treating AD associated neurodegeneration can be demonstrated in a murine model of AD. Specifically, the ginsenoside compounds (20S)Rg3, Rk1, Rg5 and Rgk351 can be used to treat mice suffering from AD associated neurodegeneration.

Mice expressing human APP as well as mice expressing the Swedish familial Alzheimer's disease mutant form of APP can be obtained from the Jackson Laboratory, 600 Main Street, Bar Harbor, Me. 04609. Four groups of mice can then be studied: (1) APP mice without ginsenoside treatment (placebo); (2) Swedish mice without ginsenoside treatment (placebo); (3) APP mice+Rg5 (100 μg/μl/day); and (4) Swedish mice+Rg5 (100 μg/μl/day). After approximately 16 weeks of injection therapy, amounts of Aβ42 in the serum of the mice can be measured. It is expected that the results of this study will demonstrate the general benefits of ginsenoside therapy for treating AD associated neuordegeneration. APP and Swedish mice without ginsenoside treatment should have significantly higher levels of serum Aβ42 and demonstrate behavior characterisitic of neurodegeneration, as compared with APP and Swedish mice receiving ginsenoside treatment.

### Example 3

The genuine sapogenines of the ginseng glycosides are structurally similar to some chemical constituents of other plants. Betulafolienetriol [dammar-24-ene-3α,12β,20(S)-triol}] isolated from birch leaves differ from the genuine sapogenin of ginseng glycosides, 20(S)-protopanaxadiol, in the configuration at C-3 only. Therefore, betulafolienetriol, cheap and relatively accesable, makes a desirable sustrate to prepare 20(S)-protopanaxadiol and its glycoside Rg3, Rg5, and Rk1.

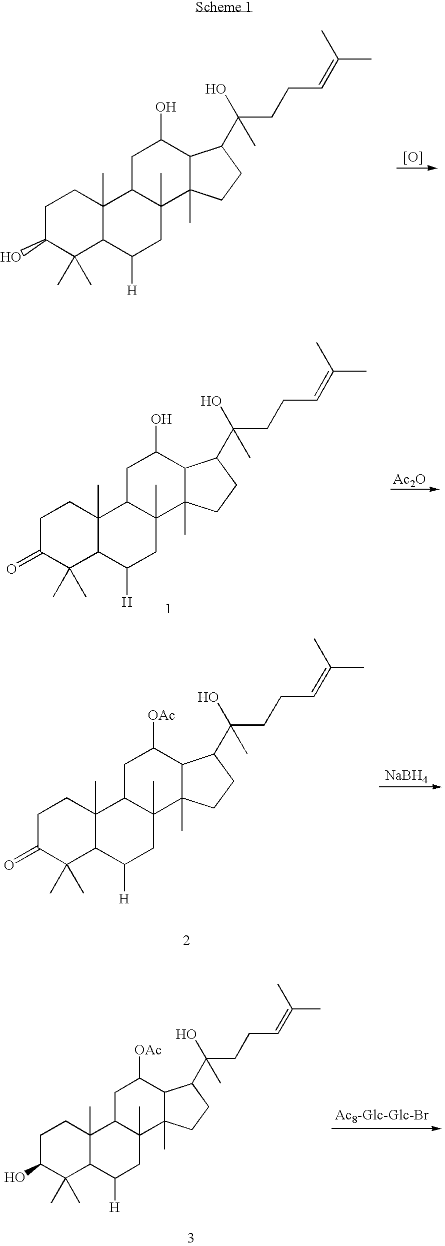

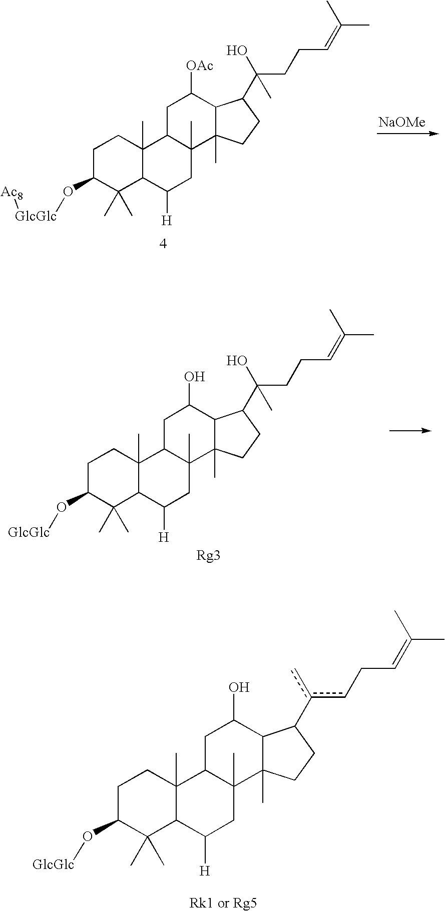

Betulafolienetriol was isolated from an ethereal extract of the leaves Btula pendula, followed by chromatography on silica gel and crystallization from acetone: mp 195-195°, lit. 197-198° (Fischer et al. (1959) Justus Liebigs Ann. Chem. 626:185).

The 12-O-acetyl derivative of 20(S)-protopanaxadiol (3) is prepared from betulafolienetriol by the sequence of reactions showen in Scheme 1. Betulafolienetriol is oxidized to ketone 1, dammar-24-ene-12β, 20(S)-diol-3-one, mp 197-199°, lit 196-199°, (yield: 60%), which is acetylated with acetic anhydride in pyridine to give compound 2, 12-O-Acetyl-dammar-24-ene-12β, 20(S)-diol-3-one (yield: 100%?) (Nagal et al., (1973) Chem. Pharm. Bull. 9:2061). ¹H NMR (CDCl₃) of the compound 2: 0.90 (s, 3H), 0.95 (s, 3H), 1.0 (s, 6H), 1.1 (s, 3H), 1.1 (s, 3H), 1.65 (s, 3H), 1.72 (s, 3H), 2.1 (s, 3H), 3.04 (s, 1H), 4.73 (td, 1H), 5.17 (t, 1H). Sodium borohydride reduction of the compound 2 in 2-propanol affords compound 3,12-O-Acetyl-dammar-24-ene-3β, 12β, 20(S)-triol (yield: 90%). 1H NMR (CDCl₃) of the compound 3: 0.78 (s, 3H), 0.86 (8, 3H), 0.95 (s, 3H), 1.0 (s, 3H), 1.02 (s, 3H), 1.13 (s, 3H), 1.64 (s, 3H), 1.71 (s, 3H), 2.05 (s, 3H, OAc), 3.20 (dd, 1H, H-3α), 4.73 (td, 1H, H-12° C.), 5.16 (t, 1H, H-24).

Condensation of compound 3 with O-acetylate-sugar bromide in the presence of silver oxide and molecular sieves 4A in dichloroethane results in formation of compound 4 (yield: 50%). Specifically, a mixture of compound 3 (1.08 g, 2 mmol), silver oxide (1.4 g, 6 mmol), α-acetobromoglucose (2.47 g, 6 mmol), molecular sieves 4A (1.0 g) and dichloroethane (20 ml) was agitated at ambient temperature until the acetobromoglucose had reacted (TLC). The reaction mixture was then diluted with CHCl₃ and filtered. The solvent was evaporated and the residue was washed with hot water to remove the excess of glucose derivatives. Silica gel column chromatography (8:1 n-hexane-acetone) gave compound 4 (853 mg). Deprotection of the glucoside 4 gives ginsenoside Rg3 which is concerted to Rk1 or Rg5 in 2 steps.

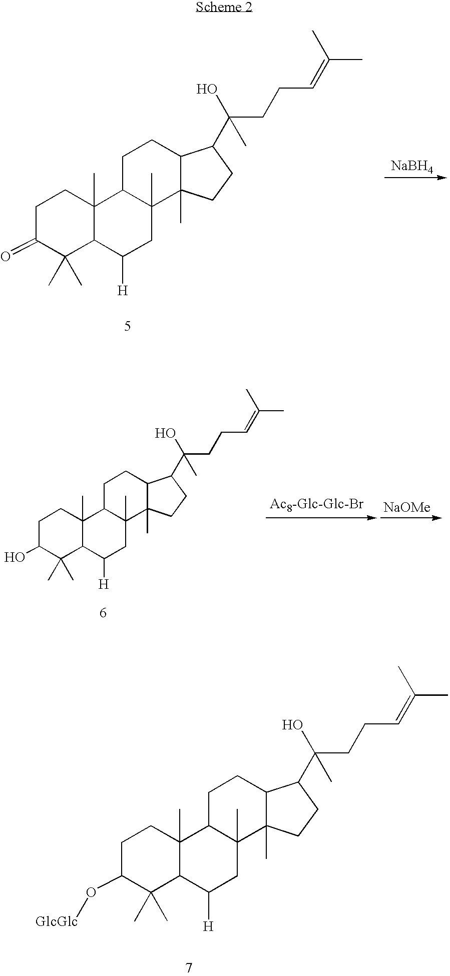

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A compound having the general formula: 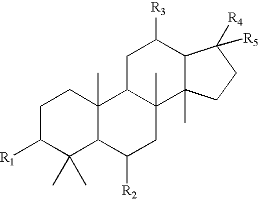
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof, R₃ is selected from the group consisting of H, OH, and OAc; R₄ is alkenyl, aryl, or alkyl II; and R₅ is H or OH.

2. The compound of claim 1, wherein the alkyl I group further contains oxygen, nitrogen, or phosphorus.

3. The compound of claim 1, wherein the alkyl II group further contains a function group selected from the group consisting of hydroxyl, ether, ketone, oxime, hydrazone, imine, and Schiff base.

4. The compound of claim 1, wherein the sugar is selected from the group consisting of Glc, Ara(pyr), Ara(fur), Rha, and Xyl.

5. The compound of claim 1, wherein the R₄ is selected from the group consisting of: 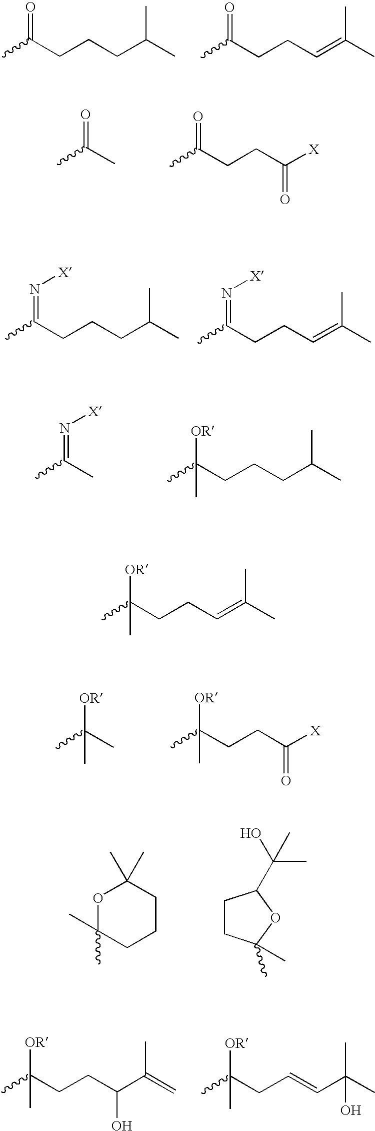
wherein the configuration of any stereo-center is R or S; X is OR or NR, wherein R is alkyl or aryl; X′ is alkyl, OR, NR, wherein R is alkyl or aryl; and R′ is H, alkyl, or acyl.

6. Use of a compound having the general formula: 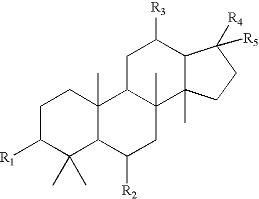
in the treatment or prevention of a pathological condition, wherein R₁ is selected from the group consisting of α-OH, β-OH, α-O—X, β-O—X, α-R₆COO—, β-R₆COO—, α-R₆PO₃—, and —R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

7. The use of claim 6, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

8. The use of claim 6, wherein the pathological condition is neurodegeneration.

9. The use of claim 8, wherein the pathological condition is Alzheimer's disease.

10. The use of claim 6, wherein the pathological condition is an Aβ42-related disorder.

11. An isolated compound having the general formula: 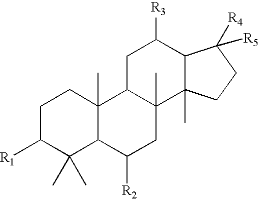
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

12. The isolated compound of claim a10, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

13. A composition comprising a compound having the general formula: 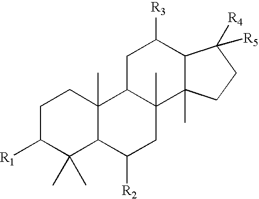
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

14. The composition of claim 13, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

15. A pharmaceutical composition comprising a pharmaceutically acceptable carrier and a compound having the general formula: 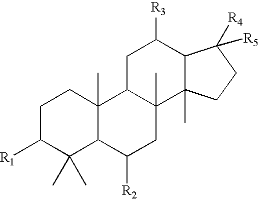
wherein R₁ is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

16. The pharmaceutical composition of claim 15, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

17. A method for the synthesis of a compound having formula: 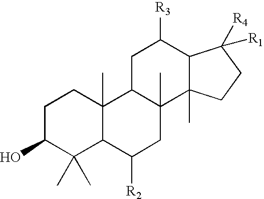
said method comprising the steps of:
(a) treating a compound having formula: 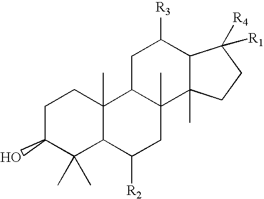
with an oxidizing agent, to form a compound having formula: 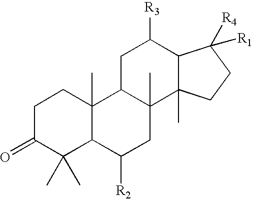
(b) treating the compound formed in step (a) with a reducing agent, to form a compound having formula: 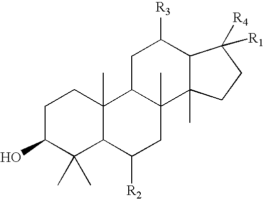
wherein R₁ is H or OH; R₂ is selected from the group consisting of H, OH, OAc, and O—X, wherein X is a carbohydrate containing one or more sugars or acylated derivatives thereof; R₃ is selected from the group consisting of H, OH, and OAc; and R₄ is alkenyl, aryl, or alkyl.

18. The method of claim 17, wherein the oxidizing agent is chromic anhydride.

19. The method of claim 17, wherein the reducing agent is NaBH₄.

20. The method of claim 17, wherein the compound having formula: 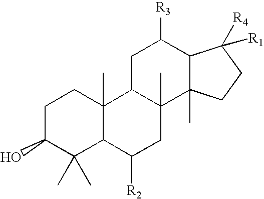
is obtained from plant.

21. The method of claim 20, wherein the plant is selected from the group consisting of common birch.

22. The method of claim 20, wherein the compound having formula: 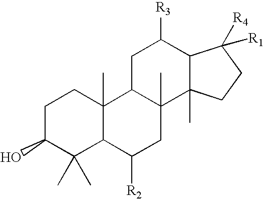
is betulafolienetriol.

23. A method for the synthesis of a compound having formula: 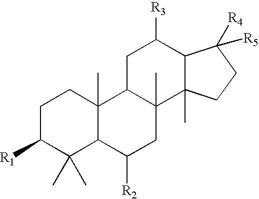
said method comprising the steps of:
(a) treating a compound having formula: 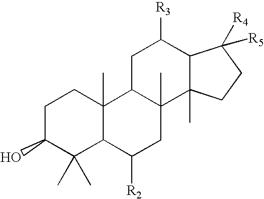
with an oxidizing agent, to form a compound having formula: 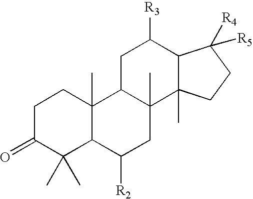
(b) treating the compound formed in step (a) with a reducing agent, to form a compound having formula: 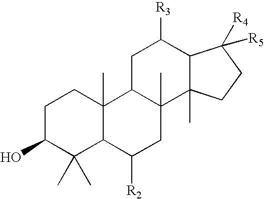
(c) optionally, treating the compound formed in step (b) with protected R₁ derivative, to form a compound having formula: 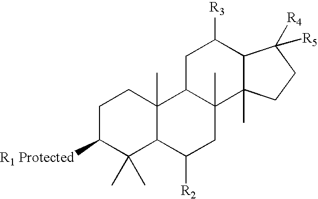
(d) treating the compound formed in step (c) with deprotection agent, to form a compound having formula: 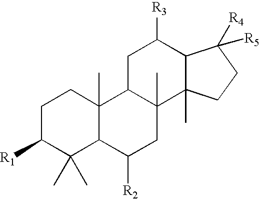
wherein R1 is selected from the group consisting of α-OH, β-OH, α-O—X, β-O—X, α-X—R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

24. The method of claim 23, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

25. The method of claim 23, wherein the oxidizing agent is chromic anhydride.

26. The method of claim 23, wherein the reducing agent is NaBH₄.

27. The method of claim 23, wherein the protected R₁ derivative is a protected R₁ halogen derivative.

28. The method of claim 23, wherein the protected R₁ derivative is protected by an Ac₈-group.

29. The method of claim 28, wherein the compound is deprotected using NaOMe.

30. The method of claim 23, wherein the compound having formula: 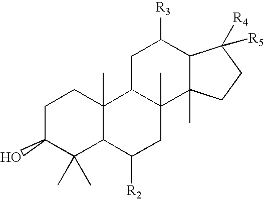
is obtained from plant.

31. The method of claim 30, wherein the plant is selected from the group consisting of common birch.

32. The method of claim 30, wherein the compound having formula: 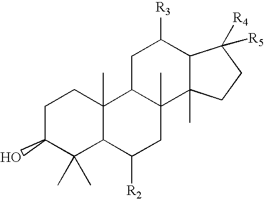
is betulafolienetriol.

33. A method for the synthesis of a compound having formula: 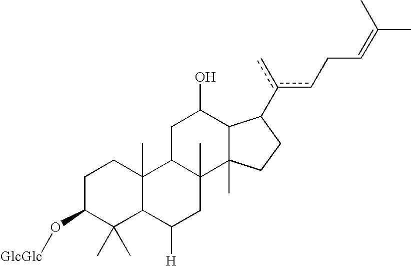
said method comprising the steps of:
(a) treating a compound having formula: 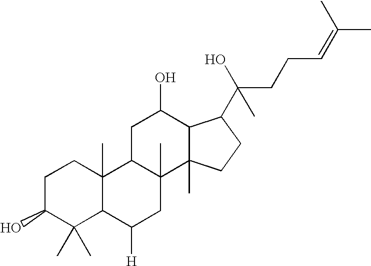
with an oxidizing agent, to form a compound having formula: 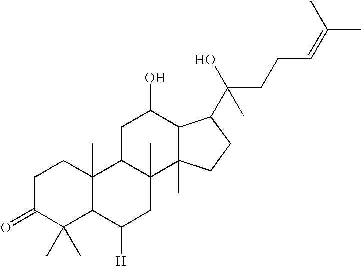
(b) treating the compound formed in step (a) with a protecting agent, to form a compound having formula: 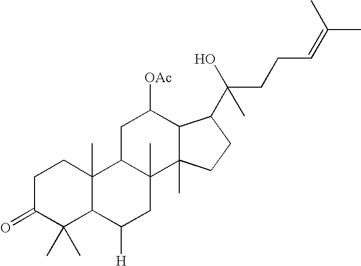
(c) treating the compound formed in step (b) with a reducing agent, to form a compound having formula: 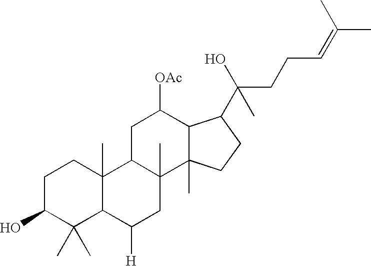
(d) treating the compound formed in step (c) with Ac₈-Glc-Glc-Br, to form a compound having formula: 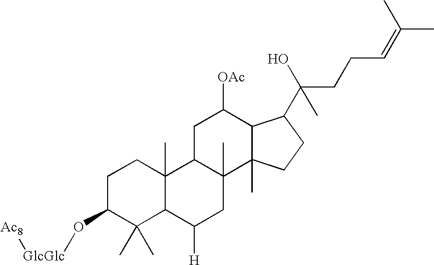
(e) treating the compound formed in step (d) with deprotection agent, to form a compound having formula: 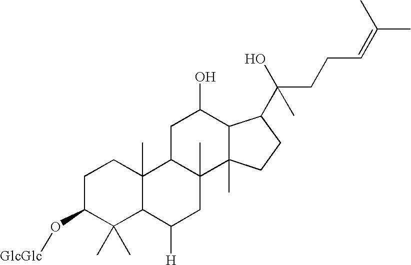
(f) further modifying the compound formed in step (e) to form a compound having formula:

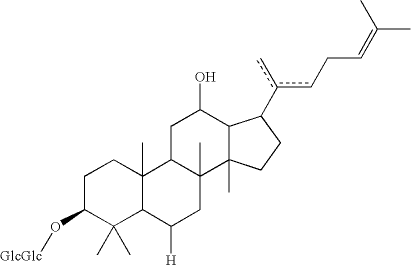

34. The method of claim 33, wherein the oxidizing agent is chromic anhydride.

35. The method of claim 33, wherein the reducing agent is NaBH₄.

36. The method of claim 33, wherein the compound is deprotected using NaOMe.

37. The method of claim 33, wherein the compound having formula: 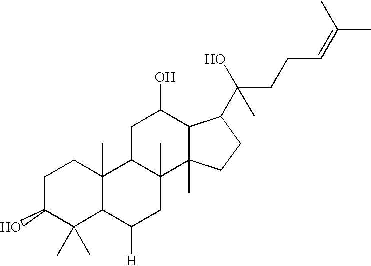
is obtained from plant.

38. The method of claim 37, wherein the plant is selected from the group consisting of common birch.

39. A method for the synthesis of a compound having formula: 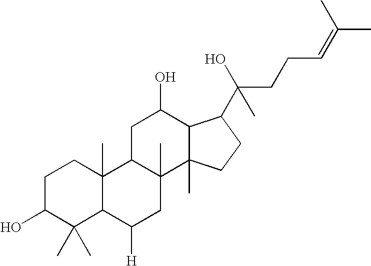
said method comprising the step of treating a compound having formula: 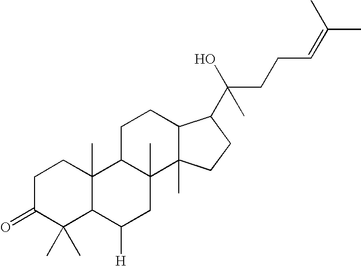
with a reducing agent, to form a compound having formula:

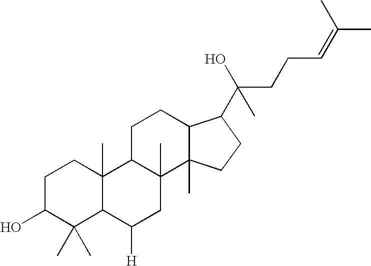

40. The method of claim 39, wherein the reducing agent is NaBH₄.

41. A method for the synthesis of a compound having formula: 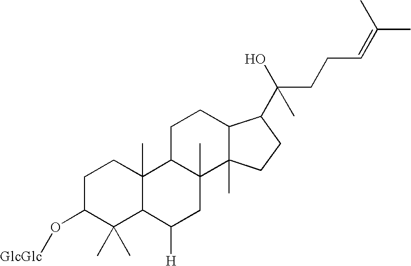
said method comprising the steps of:
(a) treating a compound having formula: 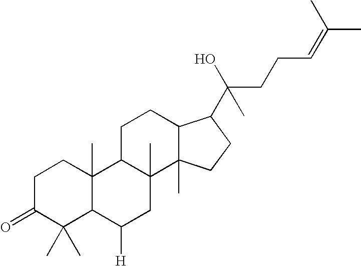
with a reducing agent, to form a compound having formula: 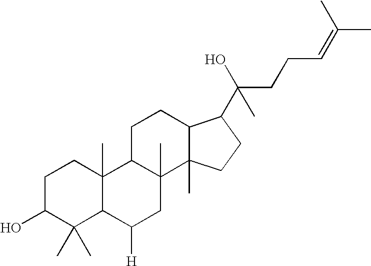
(b) treating the compound formed in step (a) with Ac₈-Glc-Glc-Br, to form a compound having formula: 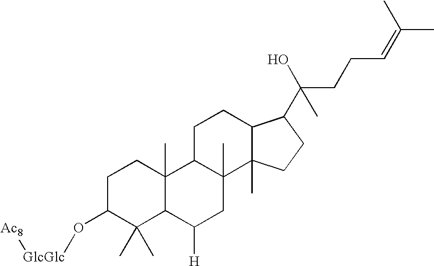
(c) treating the compound formed in step (d) with deprotection agent, to form a compound having formula:

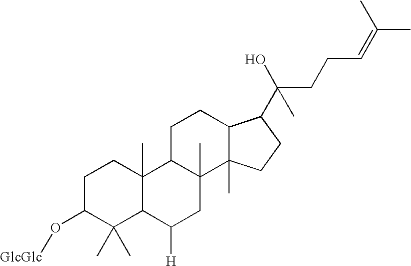

42. The method of claim 41, wherein the reducing agent is NaBH₄.

43. The method of claim 41, wherein the compound is deprotected using NaOMe.

44. A method for treating or preventing a pathological condition in a subject, comprising administering a compound having the general formula: 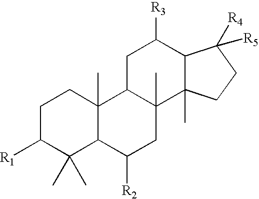
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof, R₃ is selected from the group consisting of H, OH, and OAc; R₄ is alkenyl, aryl, or alkyl II; and R₅ is H or OH.

45. The method of claim 44, wherein the alkyl I group further contains oxygen, nitrogen, or phosphorus; and the alkyl II group further contains a function group selected from the group consisting of hydroxyl, ether, ketone, oxime, hydrazone, imine, and Schiff base.

46. The method of claim 44, wherein the pathological condition is neurodegeneration.

47. The method of claim 44, wherein the pathological condition is Alzheimer's disease.

48. The method of claim 44, wherein the pathological condition is an Aβ42-related disorder.

49. The method of claim 44, wherein the subject is a human.

50. A method for inhibiting β-amyloid production in a subject, comprising administering a compound having the general formula: 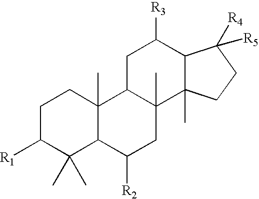
to the subject, wherein R1 is selected from the group consisting of α-OH, β-OH, α-O—X, β-O—X, α-R₆COO—, β-R₆COO—, α-R₆PO₃—, and β-R₆PO₃—, wherein X is a carbohydrate containing one or more sugars or acylated derivatives thereof, and R₆ is alkenyl, aryl, or alkyl I; R₂ is selected from the group consisting of H, OH, OAc, and O—X, wherein X is a carbohydrate containing one or more sugars or acylated derivatives thereof; R₃ is selected from the group consisting of H, OH, and OAc; R₄ is alkenyl, aryl, or alkyl II; and R₅ is H or OH.

51. The method of claim 50, wherein the alkyl I group further contains oxygen, nitrogen, or phosphorus; and the alkyl II group further contains a function group selected from the group consisting of hydroxyl, ether, ketone, oxime, hydrazone, imine, and Schiff base.
